SEQUENCE LISTING (50 sequences)

SEQ ID NO: 1 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 2 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 3 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 4 - protein, 400 aa, Homo sapiens
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL

SEQ ID NO: 5 - protein, 105 aa, Homo sapiens
MVKQIESKTAFQEALDAAGDKLVVVDFSATWCGPCKMINPFFHSLSEKYSNVIFLEVDVDDCQDVASECEVKCTPTFQFFKKGQKVGEFSGANKEKLEATINELV

SEQ ID NO: 6 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 7 - protein, 83 aa, Homo sapiens
MRLFLSLPVLVVVLSIVLEGPAPAQGTPDVSSALDKLKEFGNTLEDKARELISRIKQSELSAKMREWFSETFQKVKEKLKIDS

SEQ ID NO: 8 - protein, 261 aa, Homo sapiens
MASPDWGYDDKNGPEQWSKLYPIANGNNQSPVDIKTSETKHDTSLKPISVSYNPATAKEIINVGHSFHVNFEDNDNRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 9 - protein, 1208 aa, Homo sapiens
MEPLLLGRGLIVYLMFLLLKFSKAIEIPSSVQQVPTIIKQSKVQVAFPFDEYFQIECEAKGNPEPTFSWTKDGNPFYFTDHRIIPSNNSGTFRIPNEGHISHFQGKYRCFASNKLGIAMSEEIEFIVPSVPKFPKEKIDPLEVEEGDPIVLPCNPPKGLPPLHIYWMNIELEHIEQDERVYMSQKGDLYFANVEEKDSRNDYCCFAAFPRLRTIVQKMPMKLTVNSSNSIKQRKPKLLLPPTESGSESSITILKGEILLLECFAEGLPTPQVDWNKIGGDLPKGREAKENYGKTLKIENVSYQDKGNYRCTASNFLGTATHDFHVIVEEPPRWTKKPQSAVYSTGSNGILLCEAEGEPQPTIKWRVNGSPVDNHPFAGDVVFPREISFTNLQPNHTAVYQCEASNVHGTILANANIDVVDVRPLIQTKDGENYATVVGYSAFLHCEFFASPEAVVSWQKVEEVKPLEGRRYHIYENGTLQINRTTEEDAGSYSCWVENAIGKTAVTANLDIRNATKLRVSPKNPRIPKLHMLELHCESKCDSHLKHSLKLSWSKDGEAFEINGTEDGRIIIDGANLTISNVTLEDQGIYCCSAHTALDSAADITQVTVLDVPDPPENLHLSERQNRSVRLTWEAGADHNSNISEYIVEFEGNKEEPGRWEELTRVQGKKTTVILPLAPFVRYQFRVIAVNEVGRSQPSQPSDHHETPPAAPDRNPQNIRVQASQPKEMIIKWEPLKSMEQNGPGLEYRVTWKPQGAPVEWEEETVTNHTLRVMTPAVYAPYDVKVQAINQLGSGPDPQSVTLYSGEDYPDTAPVIHGVDVINSTLVKVTWSTVPKDRVHGRLKGYQINWWKTKSLLDGRTHPKEVNILRFSGQRNSGMVPSLDAFSEFHLTVLAYNSKGAGPESEPYIFQTPEGVPEQPTFLKVIKVDKDTATLSWGLPKKLNGNLTGYLLQYQIINDTYEIGELNDINITTPSKPSWHLSNLNATTKYKFYLRACTSQGCGKPITEESSTLGEGSKGIGKISGVNLTQKTHPVEVFEPGAEHIVRLMTKNWGDNDSIFQDVIETRGREYAGLYDDISTQGWFIGLMCAIALLTLLLLTVCFVKRNRGGKYSVKEKEDLHPDPEIQSVKDETFGEYSDSDEKPLKGSLRSLNRDMQPTESADSLVEYGEGDHGLFSEDGSFIGAYAGSKEKGSVESNGSSTATFPLRA

SEQ ID NO: 10 - protein, 163 aa, Homo sapiens
MSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 11 - protein, 2040 aa, Homo sapiens [sequence omitted]

SEQ ID NO: 12 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 13 - protein, 212 aa, Homo sapiens
MKVFKFIGLMILLTSAFSAGSGQSPMTVLCSIDWFMVTVHPFMLNNDVCVHFHELHLGLGCPPNHVQPHAYQFTYRVTECGIRAKAVSQDMVIYSTEIHYSSKGTPSKFVIPVSCAAPQKSPWLTKPCSMRVASKSRATAQKDEKCYEVFSLSQSSQRPNCDCPPCVFSEEEHTQVPCHQAGAQEAQPLQPSHFLDISEDWSLHTDDMIGSM

SEQ ID NO: 14 - protein, 7 aa, Artificial Sequence
  Target peptide sequence of S100A8
MLTELEK

SEQ ID NO: 15 - protein, 11 aa, Artificial Sequence
  Target peptide sequence of S100A8
ALNSIIDVYHK

SEQ ID NO: 16 - protein, 7 aa, Artificial Sequence
  Target peptide sequence of S100A9
DLQNFLK

SEQ ID NO: 17 - protein, 8 aa, Artificial Sequence
  Target peptide sequence of S100A9
LTWASHEK

SEQ ID NO: 18 - protein, 15 aa, Artificial Sequence
  Target peptide sequence of S100A9
NIETIINTFHQYSVK

SEQ ID NO: 19 - protein, 8 aa, Artificial Sequence
  Target peptide sequence of ANXA2
DLYDAGVK

SEQ ID NO: 20 - protein, 9 aa, Artificial Sequence
  Target peptide sequence of ANXA2
QDIAFAYQR

SEQ ID NO: 21 - protein, 13 aa, Artificial Sequence
  Target peptide sequence of KRT19
DYSHYYTTIQDLR

SEQ ID NO: 22 - protein, 13 aa, Artificial Sequence
  Target peptide sequence of KRT19
AALEDTLAETEAR

SEQ ID NO: 23 - protein, 13 aa, Artificial Sequence
  Target peptide sequence of TRX1
TAFQEALDAAGDK

SEQ ID NO: 24 - protein, 9 aa, Artificial Sequence
  Target peptide sequence of TRX1
CMPTFQFFK

SEQ ID NO: 25 - protein, 8 aa, Artificial Sequence
  Target peptide sequence of GSN
TGAQELLR

SEQ ID NO: 26 - protein, 9 aa, Artificial Sequence
  Target peptide sequence of GSN
YIETDPANR

SEQ ID NO: 27 - protein, 10 aa, Artificial Sequence
  Target peptide sequence of APOC1
TPDVSSALDK

SEQ ID NO: 28 - protein, 9 aa, Artificial Sequence
  Target peptide sequence of APOC1
EFGNTLEDK

SEQ ID NO: 29 - protein, 9 aa, Artificial Sequence
  Target peptide sequence of APOC1
EWFSETFQK

SEQ ID NO: 30 - protein, 7 aa, Artificial Sequence
  Target peptide sequence of APOC1
QSELSAK

SEQ ID NO: 31 - protein, 9 aa, Artificial Sequence
  Target peptide sequence of CA1
VLDALQAIK

SEQ ID NO: 32 - protein, 12 aa, Artificial Sequence
  Target peptide sequence of CA1
ADGLAVIGVLMK

SEQ ID NO: 33 - protein, 11 aa, Artificial Sequence
  Target peptide sequence of CHL1
IIPSNNSGTFR

SEQ ID NO: 34 - protein, 9 aa, Artificial Sequence
  Target peptide sequence of CHL1
VIAVNEVGR

SEQ ID NO: 35 - protein, 11 aa, Artificial Sequence
  Target peptide sequence of CHL1
GDLYFANVEEK

SEQ ID NO: 36 - protein, 9 aa, Artificial Sequence
  Target peptide sequence of CHL1
IENVSYQDK

SEQ ID NO: 37 - protein, 13 aa, Artificial Sequence
  Target peptide sequence of CHL1
YHIYENGTLQINR

SEQ ID NO: 38 - protein, 14 aa, Artificial Sequence
  Target peptide sequence of FN1
SYTITGLQPGTDYK

SEQ ID NO: 39 - protein, 10 aa, Artificial Sequence
  Target peptide sequence of FN1
TYHVGEQWQK

SEQ ID NO: 40 - protein, 16 aa, Artificial Sequence
  Target peptide sequence of FN1
HEEGHMLNCTCFGQGR

SEQ ID NO: 41 - protein, 10 aa, Artificial Sequence
  Target peptide sequence of FN1
STTPDITGYR

SEQ ID NO: 42 - protein, 10 aa, Artificial Sequence
  Target peptide sequence of LPA
WVLTAAHCLK

SEQ ID NO: 43 - protein, 10 aa, Artificial Sequence
  Target peptide sequence of LPA
GTYSTTVTGR

SEQ ID NO: 44 - protein, 9 aa, Artificial Sequence
  Target peptide sequence of LPA
YICAEHLAR

SEQ ID NO: 45 - protein, 13 aa, Artificial Sequence
  Target peptide sequence of LPA
NPDAVAAPYCYTR

SEQ ID NO: 46 - protein, 9 aa, Artificial Sequence
  Target peptide sequence of MUC1
YVPPSSTDR

SEQ ID NO: 47 - protein, 12 aa, Artificial Sequence
  Target peptide sequence of PLAC1
FVIPVSCAAPQK

SEQ ID NO: 48 - protein, 9 aa, Artificial Sequence
  Target peptide sequence of E. coli beta-galactosidase
GDFQFNISR

SEQ ID NO: 49 - protein, 9 aa, Artificial Sequence
  Target peptide sequence of TRX1
VGEFSGANK

SEQ ID NO: 50 - protein, 13 aa, Artificial Sequence
  Target peptide sequence of TRX1
TAFQEALDAAGDK

(19) United States
(12) Patent Application Publication
(10) Pub. No.: US 2022/0127679 A1
(43) Pub. Date: Apr. 28, 2022

(54) COMPOSITION FOR CANCER DIAGNOSIS

(71) Applicant: BERTIS CO., LTD., Seongnam-si, Gyeonggi-do (KR)

(72) Inventors: Sung Soo KIM, Yongin-si (KR); Seung Man HAN, Seoul (KR)

(73) Assignee: BERTIS INC, Seongnam-si, Gyeonggi-do (KR)

(21) Appl. No.: 17/430,378

(22) PCT Filed: Feb. 13, 2020
(86) PCT No.: PCT/KR2020/002031

§ 371 Date: Aug. 12, 2021

(30) Foreign Application Priority Data
Feb. 13, 2019 (KR) 10-2019-0016654

(51) Int. Cl. C12Q 1/6886

(52) U.S. Cl. CPC C12Q 1/6886; C12Q 2600/158

(57) ABSTRACT

The present disclosure relates to a composition capable of cancer diagnosis, a diagnostic kit comprising same, and a method for providing information for cancer diagnosis using the composition. When a biomarker of the present disclosure is used, it is possible to accurately and conveniently diagnose cancer, particularly breast cancer, in an early stage, and furthermore, it is possible to diagnose the stage of cancer and predict therapeutic responsiveness or post-treatment prognosis.

### TECHNICAL FIELD

The present disclosure relates to a composition capable of diagnosing cancer, a diagnostic kit comprising the same, and a method of providing information for cancer diagnosis using the composition.

### BACKGROUND ART

Breast cancer is the second most common cancer after lung cancer in the world, and is a dangerous cancer that ranks fifth in mortality rate. In the case of breast cancer, once cancer cells invade surrounding tissues or start to metastasize to lymph nodes, it is difficult to cure breast cancer. Thus, it can be said that early diagnosis of breast cancer is more important than other cancers.

In order to reduce the mortality rate caused by breast cancer, it is important to (1) diagnose breast cancer early, and (2) predict the prognosis after treatment by primary surgery and perform appropriate adjuvant therapy. Currently, for the diagnosis of breast cancer, in addition to self-diagnosis by primary palpation, mammography, ultrasonography, etc. are used as medical examination methods for preventive purposes. These methods are also most widely used to diagnose early breast cancer. However, mammography has a disadvantage in that the diagnosis rate of breast cancer is low in the case of dense breasts commonly found in Korean women because the breasts have a lot of fibers, and the diagnosis rate is also low, particularly in the case of young women with well-developed mammary glands. In addition, due to the use of X-rays, the likelihood of developing breast cancer during the diagnosis process cannot be excluded. Hence, ultrasonography is used as an alternative, but even with this method, it is difficult to distinguish between malignant tumors (cancer) and benign tumors (non-cancer). In actual clinical practice, if there are abnormal findings, fine needle aspiration cytology, magnetic resonance imaging, etc. are additionally used to increase the diagnosis rate. However, even with these methods, it is merely possible to morphologically distinguish between normal tissue and abnormal tissue, and it is difficult to distinguish between malignant tumors (cancer) and benign tumors (non-cancer). For confirmation of breast cancer, a more precise biopsy is performed.

For these reasons, methods of diagnosing breast cancer by a molecular genetic method have been much more developed than other cancers. In biopsy, the tissue is cut to confirm the lesion, and primary surgery for excision is performed. To determine how to conduct subsequent adjuvant therapy, a method is used, which determines the presence or absence of estrogen receptor (ER) and the number of HER2/neu (Human Epidermal growth factor Receptor 2, also known as ErbB-2 or ERBB2) gene, which is a breast cancer-specific tumor marker, by in situ hybridization. If the detected cancer tissue has an estrogen receptor, treatment is performed using an estrogen analogue such as tamoxifen, and if the HER2/neu gene is overexpressed, the HER2/neu monoclonal antibody trastuzumab commercially available under the trade name Herceptin is used for the treatment of breast cancer Amplification and overexpression of the HER2/neu gene, which is a breast cancer-specific diagnostic marker most useful for diagnosis and treatment of breast cancer, is found in 20 to 35% of invasive breast cancer. Thus, the HER2/neu test together with the estrogen receptor test plays a crucial role in the treatment of breast cancer patients.

However, since not all breast cancer patients overexpress the ER gene or Her2/neu gene, various attempts have been made to find cancer-specific biomarkers in order to overcome the shortcomings of these diagnostic methods and to detect cancer more quickly and reliably.

### DISCLOSURE

### Technical Problem

An object of the present disclosure is to provide a composition capable of accurately and conveniently diagnosing cancer, particularly breast cancer.

Another object of the present disclosure is to provide a kit capable of accurately and conveniently diagnosing cancer, particularly breast cancer.

Still another object of the present disclosure is to provide a method for providing information for diagnosing cancer, particularly breast cancer.

Yet another object of the present disclosure is to provide a method for predicting the therapeutic responsiveness of cancer, particularly breast cancer.

Still yet another object of the present disclosure is to provide a method for predicting the prognosis of cancer, particularly breast cancer.

A further object of the present disclosure is to provide a method for predicting the stage of cancer, particularly breast cancer.

Another further object of the present disclosure is to provide a method for predicting the likelihood of recurrence of cancer, particularly breast cancer.

Still another further object of the present disclosure is to provide a method for screening a drug for treating cancer, particularly breast cancer.

However, objects to be achieved by the present disclosure are not limited to the objects mentioned above, and other objects not mentioned herein will be clearly understood by those of ordinary skill in the art from the following description.

### Technical Solution

According to one embodiment of the present disclosure, the present disclosure is directed to a cancer diagnostic marker comprising at least one selected from the group consisting of S100A8, S100A9, ANXA2, KRT19, TRX1, GSN, APOC1, CA1, CHL1, FN1, LPA, MUC1 and PLAC1.

In the present disclosure, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

According to another embodiment of the present disclosure, the present disclosure is directed to a cancer diagnostic composition containing an agent for measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein.

In the present disclosure, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

In the present disclosure, the agent for measuring the expression level of the S100A8, S100A9, ANXA2, KRT19, TRX1 GSN, APOC1, CA1, CHL1, FN1, LPA, MUC1 or PLAC1 protein may comprise at least one selected from the group consisting of an antibody, an oligopeptide, a ligand, a peptide nucleic acid (PNA) and an aptamer, which bind specifically to the S100A8, S100A9, ANXA2, KRT19, TRX1, GSN, APOC1, CA1, CHL1, FN1, LPA, MUC1 or PLAC1 protein.

In the present disclosure, the agent for measuring the expression level of the gene encoding the S100A8, S100A9, ANXA2, KRT19, TRX1 GSN, APOC1, CA1, CHL1, FN1, LPA, MUC1 or PLAC1 protein may comprise at least one selected from the group consisting of a primer, a probe and an antisense nucleotide, which bind specifically to the gene encoding the S100A8, S100A9, ANXA2, KRT19, TRX1, GSN, APOC1, CA1, CHL1, FN1, LPA, MUC1 or PLAC1 protein.

According to still another embodiment of the present disclosure, the present disclosure is directed to a cancer diagnostic kit comprising the cancer diagnostic composition according to the present disclosure.

In the present disclosure, the kit may be an RT-PCR kit, a DNA chip kit, an ELISA kit, a protein chip kit, a rapid kit or a multiple-reaction monitoring (MRM) kit.

According to yet another embodiment of the present disclosure, the present disclosure is directed to a method for providing information for cancer diagnosis, the method comprising a step of measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in a biological sample isolated from a subject of interest.

In the present disclosure, the biological sample may be whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid.

In the present disclosure, an agent for measuring the expression level of the S100A8, S100A9, ANXA2, KRT19, TRX1 GSN, APOC1, CA1, CHL1, FN1, LPA, MUC1 or PLAC1 protein may comprise at least one selected from the group consisting of an antibody, an oligopeptide, a ligand, a peptide nucleic acid (PNA) and an aptamer, which bind specifically to the S100A8, S100A9, ANXA2, KRT19, TRX1, GSN, APOC1, CA1, CHL1, FN1, LPA, MUC1 or PLAC1 protein.

In the present disclosure, the measurement of the expression level of the S100A8, S100A9, ANXA2, KRT19, TRX1, GSN, APOC1, CA1, CHL1, FN1, LPA, MUC1 or PLAC1 protein may be performed by protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoelectrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, or ELISA (enzyme-linked immunosorbent assay).

In the present disclosure, the measurement of the expression level of the S100A8, S100A9, ANXA2, KRT19, TRX1, GSN, APOC1, CA1, CHL1, FN1, LPA, MUC1 or PLAC1 protein may be performed by a multiple-reaction monitoring (MRM) method.

In the present disclosure, the target peptide of the S100A8 may consist of the amino acid sequence represented by SEQ ID NO: 14 or 15; or

the target peptide of the S100A9 may consist of the amino acid sequence represented by any one of SEQ ID NOs: 16 to 18; or

the target peptide of the ANXA2 may consist of the amino acid sequence represented by SEQ ID NO: 19 or 20; or

the target peptide of the KRT19 may consist of the amino acid sequence represented by SEQ ID NO: 21 or 22; or

the target peptide of the TRX1 may consist of the amino acid sequence represented by SEQ ID NO: 23, 24, 49 or 50; or

the target peptide representing the GSN may consist of the amino acid sequence represented by SEQ ID NO: 25 or 26; or

the target peptide representing the APOC1 may consist of the amino acid sequence represented by any one of SEQ ID NOs: 27 to 30; or

the target peptide representing the CA1 may consist of the amino acid sequence represented by SEQ ID NO: 31 or 32; or

the target peptide representing the CHL1 may consist of the amino acid sequence represented by any one of SEQ ID NOs: 33 to 37; or

the target peptide representing the FN1 may consist of the amino acid sequence represented by any one of SEQ ID NOs: 38 to 41; or

the target peptide representing the LPA may consist of the amino acid sequence represented by any one of SEQ ID NOs: 42 to 45; or

the target peptide representing the MUC1 may consist of the amino acid sequence represented by SEQ ID NO: 46; or

the target peptide representing the PLAC1 may consist of the amino acid sequence represented by SEQ ID NO: 47.

In the present disclosure, a pair of mother and daughter ions of the target peptide of the S100A8 may have mass-to-charge ratios of 432.23 m/z and 732.41 m/z, 432.23 m/z and 619.33 m/z, 432.23 m/z and 518.28 m/z, 636.85 m/z and 887.50 m/z, 636.85 m/z and 774.41 m/z, 636.85 m/z and 661.33 m/z, or 636.85 m/z and 546.30 m/z, respectively; or

a pair of mother and daughter ions of the target peptide of the S100A9 may have mass-to-charge ratios of 439.24 m/z and 649.37 m/z, 439.24 m/z and 521.31 m/z, 439.24 m/z and 407.27 m/z, 486.25 m/z and 757.36 m/z, 486.25 m/z and 571.28 m/z, 486.25 m/z and 500.25 m/z, 486.25 m/z and 413.21 m/z, 602.98 m/z and 908.46 m/z, 602.98 m/z and 761.39 m/z, 602.98 m/z and 624.34 m/z, or 602.98 m/z and 486.28 m/z, respectively; or

a pair of mother and daughter ions of the target peptide of the ANXA2 may have mass-to-charge ratios of 440.72 m/z and 652.33 m/z, 440.72 m/z and 489.27 m/z, 440.72 m/z and 374.24 m/z, 440.72 m/z and 303.20 m/z, 556.28 m/z and 868.47 m/z, 556.28 m/z and 755.38 m/z, 556.28 m/z and 684.35 m/z, 556.28 m/z and 537.28 m/z, or 556.28 m/z and 466.24 m/z, respectively; or

a pair of mother and daughter ions of the target peptide of the KRT19 may have mass-to-charge ratios of 558.93 m/z and 846.47 m/z, 558.93 m/z and 745.42 m/z, 558.93 m/z and 644.37 m/z, 558.93 m/z and 531.29 m/z, 695.35 m/z and 789.41 m/z, 695.35 m/z and 676.33 m/z, 695.35 m/z and 605.29 m/z, 695.35 m/z and 476.25 m/z, or 695.35 m/z and 375.20 m/z, respectively; or

a pair of mother and daughter ions of the target peptide of the TRX1 may have mass-to-charge ratios of 668.82 m/z and 889.43 m/z, 668.82 m/z and 760.38 m/z, 668.82 m/z and 689.35 m/z, 668.82 m/z and 576.26 m/z, 668.82 m/z and 461.24 m/z, 603.28 m/z and 914.48 m/z, 603.28 m/z and 817.42 m/z, 603.28 m/z and 716.38 m/z, 603.28 m/z and 569.31 m/z, 603.28 m/z and 441.25 m/z, 454.727 m/z and 809.379 m/z, 454.727 m/z and 752.357 m/z, 454.727 m/z and 623.315 m/z, 454.727 m/z and 476.246 m/z, 454.727 m/z and 389.214 m/z, 668.823 m/z and 889.426 m/z, 668.823 m/z and 760.384 m/z, 668.823 m/z and 689.346 m/z, or 668.823 m/z and 576.262 m/z, respectively; or

a pair of mother and daughter ions of the target peptide of the GSN may have mass-to-charge ratios of 444.25 m/z and 729.43 m/z, 444.25 m/z and 658.39 m/z, 444.25 m/z and 530.33 m/z, 444.25 m/z and 401.29 m/z, 539.76 m/z and 802.37 m/z, 539.76 m/z and 673.33 m/z, 539.76 m/z and 572.28 m/z, 539.76 m/z and 457.25 m/z, or 539.76 m/z and 360.20 m/z, respectively; or

a pair of mother and daughter ions of the target peptide of the APOC1 may have mass-to-charge ratios of 516.76 m/z and 719.39 m/z, 516.76 m/z and 620.32 m/z, 516.76 m/z and 533.29 m/z, 516.76 m/z and 466.24 m/z, 526.75 m/z and 605.31 m/z, 526.75 m/z and 776.38 m/z, 526.75 m/z and 719.36 m/z, 526.75 m/z and 504.27 m/z, 526.75 m/z and 391.18 m/z, 601.28 m/z and 886.43 m/z, 601.28 m/z and 739.36 m/z, 601.28 m/z and 652.33 m/z, 601.28 m/z and 523.29 m/z, 601.28 m/z and 422.24 m/z, 381.70 m/z and 547.31 m/z, 381.70 m/z and 418.27 m/z, or 381.70 m/z and 305.18 m/z, respectively; or

a pair of mother and daughter ions of the target peptide of the CA1 may have mass-to-charge ratios of 485.80 m/z and 758.44 m/z, 485.80 m/z and 643.41 m/z, 485.80 m/z and 572.38 m/z, 485.80 m/z and 459.29 m/z, 593.85 m/z and 759.48 m/z, 593.85 m/z and 660.41 m/z, 593.85 m/z and 547.33 m/z, or 593.85 m/z and 490.31 m/z, respectively; or

a pair of mother and daughter ions of the target peptide of the CHL1 may have mass-to-charge ratios of 603.32 m/z and 490.23 m/z, 603.32 m/z and 795.37 m/z, 603.32 m/z and 681.33 m/z, 603.32 m/z and 567.29 m/z, 603.32 m/z and 480.26 m/z, 478.78 m/z and 744.40 m/z, 478.78 m/z and 673.36 m/z, 478.78 m/z and 574.29 m/z, 478.78 m/z and 460.25 m/z, 642.81 m/z and 836.42 m/z, 642.81 m/z and 689.35 m/z, 642.81 m/z and 618.31 m/z, 642.81 m/z and 504.27 m/z, 548.27 m/z and 853.41 m/z, 548.27 m/z and 739.36 m/z, 548.27 m/z and 640.29 m/z, 548.27 m/z and 553.26 m/z, 548.27 m/z and 390.20 m/z, 540.94 m/z and 915.50 m/z, 540.94 m/z and 801.46 m/z, 540.94 m/z and 744.44 m/z, 540.94 m/z and 643.39 m/z, or 540.94 m/z and 530.30 m/z, respectively; or

a pair of mother and daughter ions of the target peptide of the FN1 may have mass-to-charge ratios of 772.39 m/z and 808.38 m/z, 772.39 m/z and 680.32 m/z, 772.39 m/z and 583.27 m/z, 772.39 m/z and 526.25 m/z, 425.88 m/z and 1011.50 m/z, 425.88 m/z and 874.44 m/z, 425.88 m/z and 775.37 m/z, 425.88 m/z and 718.35 m/z, 644.94 m/z and 985.40 m/z, 644.94 m/z and 825.37 m/z, 644.94 m/z and 724.32 m/z, 644.94 m/z and 564.29 m/z, 644.94 m/z and 417.22 m/z, 555.78 m/z and 922.46 m/z, 555.78 m/z and 821.42 m/z, 555.78 m/z and 724.36 m/z, or 555.78 m/z and 609.34 m/z, respectively; or

a pair of mother and daughter ions of the target peptide of the LPA may have mass-to-charge ratios of 400.22 m/z and 400.71 m/z, 400.22 m/z and 800.41 m/z, 400.22 m/z and 699.36 m/z, 400.22 m/z and 628.32 m/z, 400.22 m/z and 557.29 m/z, 521.76 m/z and 634.30 m/z, 521.76 m/z and 884.45 m/z, 521.76 m/z and 721.38 m/z, 521.76 m/z and 533.30 m/z, 566.78 m/z and 696.38 m/z, 566.78 m/z and 625.34 m/z, 566.78 m/z and 496.30 m/z, 566.78 m/z and 359.24 m/z, 749.34 m/z and 1171.56 m/z, 749.34 m/z and 1100.52 m/z, 749.34 m/z and 1001.45 m/z, or 749.34 m/z and 930.41 m/z, respectively;

a pair of mother and daughter ions of the target peptide of the MUC1 may have mass-to-charge ratios of 511.25 m/z and 759.36 m/z, 511.25 m/z and 662.31 m/z, 511.25 m/z and 565.26 m/z, 511.25 m/z and 478.23 m/z, or 511.25 m/z and 391.19 m/z, respectively; or

a pair of mother and daughter ions of the target peptide of the PLAC1 may have mass-to-charge ratios of 658.86 m/z and 1070.57 m/z, 658.86 m/z and 957.48 m/z, 658.86 m/z and 860.43 m/z, 658.86 m/z and 761.36 m/z, 658.86 m/z and 674.33 m/z, or 658.86 m/z and 514.30 m/z, respectively.

In the present disclosure, an internal standard substance that is used in the multiple-reaction monitoring method may be either a synthetic peptide obtained by substituting certain amino acids of the target peptide with an isotope, or E. coli beta-galactosidase.

In the present disclosure, the target peptide of the E. coli beta-galactosidase may consist of the amino acid sequence of SEQ ID NO: 48, and mother and daughter ions thereof may have mass-to-charge ratios of m/z 542.3 and m/z 636.3, respectively.

In the present disclosure, an agent for measuring the expression level of the gene encoding the S100A8, S100A9, ANXA2, KRT19, TRX1 GSN, APOC1, CA1, CHL1, FN1, LPA, MUC1 or PLAC1 protein may comprise at least one selected from the group consisting of a primer, a probe and an antisense nucleotide, which bind specifically to the S100A8, S100A9, ANXA2, KRT19, TRX1, GSN, APOC1, CA1, CHL1, FN1, LPA, MUC1 or PLAC1 protein.

In the present disclosure, the measurement of the expression level of the gene encoding the S100A8, S100A9, ANXA2, KRT19, TRX1 GSN, APOC1, CA1, CHL1, FN1, LPA, MUC1 or PLAC1 protein may be performed by reverse transcription-polymerase chain reaction (RT-PCR), competitive RT-PCR, real-time RT-PCR, RNase protection assay (RPA), Northern blotting, or DNA chip assay.

In the present disclosure, if the measured expression level of either at least one protein selected from the group consisting of S100A8, S100A9, ANXA2, KRT19, TRX1, GSN, APOC1, CA1, CHL1, FN1, LPA, MUC1 and PLAC1, or the gene encoding the protein, in the biological sample isolated from the subject of interest, increases or decreases compared to a normal control, it may be predicted that the subject has a likelihood of developing the cancer.

In the present disclosure, the method for providing information may be a method of predicting the responsiveness of the subject of interest to anticancer chemotherapy or immunotherapy.

In the present disclosure, the method for providing information may be a method of predicting the prognosis of the subject of interest after surgical operation.

In the present disclosure, the method for providing information may be a method of diagnosing the stage of cancer in the subject of interest.

In the present disclosure, the method for providing information may be a method of predicting the likelihood of recurrence of cancer in the subject of interest.

In the present disclosure, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

According to another embodiment of the present disclosure, the present disclosure is directed to a method for screening a drug for preventing or treating cancer, the method comprising steps of: (a) treating either a sample isolated from a cancer subject or a cancer disease animal model with a candidate drug; and (b) measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in the sample or cancer disease animal model treated with the candidate drug.

In the present disclosure, the sample may be cells or tissue isolated from the cancer subject.

In the present disclosure, the method may further comprise step (c) of determining that the candidate drug is the drug for preventing or treating cancer, when the expression level of the S100A8, S100A9, ANXA2, KRT19, TRX1, GSN, APOC1, CA1, CHL1, FN1, LPA, MUC1 or PLAC1 protein, or the gene encoding the protein, measured in step (b), decreases or increases compared to that before treatment with the candidate drug.

In the present disclosure, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

According to another embodiment of the present disclosure, the present disclosure is directed to a cancer diagnostic composition containing an agent for measuring the expression level of either the polypeptide represented by SEQ ID NO: 23, 24, 49 or 50, or a gene encoding the polypeptide.

In the present disclosure, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

In the present disclosure, the agent for measuring the expression level of the polypeptide represented by SEQ ID NO: 23, 24, 49 or 50 may comprise at least one selected from the group consisting of an antibody, an oligopeptide, a ligand, a peptide nucleic acid (PNA) and an aptamer, which bind specifically to the polypeptide.

In the present disclosure, the agent for measuring the expression level of the gene encoding the polypeptide represented by SEQ ID NO: 23, 24, 49 or 50 may comprise at least one selected from the group consisting of a primer, a probe and an antisense nucleotide, which bind specifically to the gene encoding the polypeptide.

According to another embodiment of the present disclosure, the present disclosure is directed to a cancer diagnostic kit comprising the cancer diagnostic composition.

In the present disclosure, the kit may be an RT-PCR kit, a DNA chip kit, an ELISA kit, a protein chip kit, a rapid kit or a multiple-reaction monitoring (MRM) kit.

According to another embodiment of the present disclosure, the present disclosure is directed to a method for providing information for cancer diagnosis, the method comprising a step of measuring the expression level of either the polypeptide represented by SEQ ID NO: 23, 24, 49 or 50, or a gene encoding the polypeptide, in a biological sample isolated from a subject of interest.

In the present disclosure, the agent for measuring the expression level of the polypeptide represented by SEQ ID NO: 23, 24, 49 or 50 may comprise at least one selected from the group consisting of an antibody, an oligopeptide, a ligand, a peptide nucleic acid (PNA) and an aptamer, which bind specifically to the polypeptide represented by SEQ ID NO: 23, 24, 49 or 50.

In the present disclosure, the measurement of the expression level of the polypeptide represented by SEQ ID NO: 23, 24, 49 or 50 may be performed by, but not limited to, protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoelectrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, or ELISA (enzyme-linked immunosorbent assay).

In the present disclosure, the measurement of the expression level of the polypeptide represented by SEQ ID NO: 23, 24, 49 or 50 may be performed by a multiple-reaction monitoring (MRM) method.

In the present disclosure, the mass-to-charge ratios of mother and daughter ions of the target peptide representing the polypeptide represented by SEQ ID NO: 23, 24, 49 or 50 may be, but are not limited to, 668.82 m/z and 889.43 m/z, 668.82 m/z and 760.38 m/z, 668.82 m/z and 689.35 m/z, 668.82 m/z and 576.26 m/z, 668.82 m/z and 461.24 m/z, 603.28 m/z and 914.48 m/z, 603.28 m/z and 817.42 m/z, 603.28 m/z and 716.38 m/z, 603.28 m/z and 569.31 m/z, 603.28 m/z and 441.25 m/z, 454.727 m/z and 809.379 m/z, 454.727 m/z and 752.357 m/z, 454.727 m/z and 623.315 m/z, 454.727 m/z and 476.246 m/z, 454.727 m/z and 389.214 m/z, 668.823 m/z and 889.426 m/z, 668.823 m/z and 760.384 m/z, 668.823 m/z and 689.346 m/z, or 668.823 m/z and 576.262 m/z, respectively.

In the present disclosure, an internal standard substance that is used in the multiple-reaction monitoring method in the method for providing information for cancer diagnosis may be either a synthetic peptide obtained by substituting certain amino acids of the target peptide with an isotope, or E. coli beta-galactosidase.

In the present disclosure, the target peptide of the E. coli beta-galactosidase may consist of the polypeptide of SEQ ID NO: 3, and mother and daughter ions thereof may have mass-to-charge ratios of 542.3 m/z and 636.3 m/z, respectively.

In the present disclosure, an agent for measuring the expression level of the gene encoding the polypeptide represented by SEQ ID NO: 23, 24, 49 or 50 may comprise at least one selected from the group consisting of a primer, a probe and an antisense nucleotide, which bind specifically to the gene encoding the polypeptide.

In the present disclosure, the measurement of the expression level of the gene encoding the polypeptide represented by SEQ ID NO: 23, 24, 49 or 50 may be performed by reverse transcription-polymerase chain reaction (RT-PCR), competitive RT-PCR, real-time RT-PCR, RNase protection assay (RPA), Northern blotting, or DNA chip assay.

According to another embodiment of the present disclosure, the present disclosure is directed to a method for providing information for cancer diagnosis, the method comprising a step of predicting that the likelihood of developing the cancer is high, when the measured expression level of either the polypeptide represented by SEQ ID NO: 23, 24, 49 or 50, or the gene encoding the polypeptide, in the biological sample isolated from the subject of interest, increases compared to a normal control.

In the present disclosure, the method for providing information may be a method of predicting the prognosis of the subject of interest after surgical operation.

In the present disclosure, the method for providing information may be a method of diagnosing the stage of cancer in the subject of interest.

In the present disclosure, the method for providing information may be a method of predicting the likelihood of recurrence of cancer in the subject of interest.

In the present disclosure, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

According to another embodiment of the present disclosure, the present disclosure is directed to a method for screening a drug for preventing or treating cancer, the method comprising steps of: (a) treating either a sample isolated from a cancer subject or a cancer disease animal model with a candidate drug; and (b) measuring the expression level of either the polypeptide represented by SEQ ID NO: 23, 24, 49 or 50, or a gene encoding the polypeptide, in the sample or cancer disease animal model treated with the candidate drug.

In the present disclosure, the sample may be cells or tissue isolated from the cancer subject.

In the present disclosure, the method may further comprise step (c) of determining that the candidate drug is the drug for preventing or treating cancer, when the expression level of either the polypeptide represented by SEQ ID NO: 23, 24, 49 or 50, or the gene encoding the polypeptide, measured in step (b), increases or decreases compared to that before treatment with the candidate drug.

In the present disclosure, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

According to another embodiment of the present disclosure, the present disclosure is directed to a diagnostic device comprising: a sample portion configured to receive a sample isolated from a patient; a detection portion configured to detect either the polypeptide represented by SEQ ID NO: 23, 24, 49 or 50, or a gene encoding the polypeptide, in the sample received in the sample portion; and a comparison portion configured to compare the expression level of the polypeptide represented by SEQ ID NO: 23, 24, 49 or 50, or the gene encoding the polypeptide, obtained from the detection portion, with the expression level of the polypeptide or the gene in a normal person, wherein cancer is diagnosed depending on the result obtained through the comparison portion.

In the diagnostic device of the present disclosure, the patient is diagnosed with breast cancer, when the polypeptide represented by SEQ ID NO: 23, 24, 49 or 50, or the gene encoding the polypeptide, is detected by the comparison portion.

### Advantageous Effects

When the biomarker of the present disclosure is used, it is possible to accurately and conveniently diagnose cancer, particularly breast cancer, in an early stage, and furthermore, it is possible to diagnose the stage of cancer and predict therapeutic responsiveness or post-treatment prognosis.

### BRIEF DESCRIPTION OF DRAWINGS

FIG. 1 shows a receiver operating characteristic (ROC) graph obtained for 66 breast cancer patients and 66 normal persons using a combination of APOC1, CA1 and CHL1 biomarkers in Example 1 of the present disclosure.

FIG. 2 shows a receiver operating characteristic (ROC) graph obtained for 66 breast cancer patients and 66 normal persons using a combination of APOC1, CA1, CHL1, S100A9 and S100A8 biomarkers in Example 1 of the present disclosure.

FIG. 3 shows a receiver operating characteristic (ROC) graph obtained for 66 breast cancer patients and 66 normal persons using a combination of APOC1, CA1, CHL1, S100A9, S100A8, ANXA2 and GSN biomarkers in Example 1 of the present disclosure.

FIG. 4 is a graph showing the results of confirming the difference in the expression level of thioredoxin-1 (TRX1) between breast cancer patients and a non-patient control group in Example 1 of the present disclosure.

FIG. 5 shows a receiver operating characteristic (ROC) graph obtained based on the results of quantifying thioredoxin-1 (TRX1) as a biomarker using the target peptide of the polypeptide represented by SEQ ID NO: 2 between breast cancer patients and a non-patient control group in Example 1 of the present disclosure.

FIG. 6 is a graph showing the results of confirming the difference in the expression level of thioredoxin-1 (TRX1) between breast cancer patients and a non-patient control group in Example 1 of the present disclosure.

FIG. 7 shows a receiver operating characteristic (ROC) graph obtained based on the results of quantifying thioredoxin-1 (TRX1) as a biomarker using the target peptide of the polypeptide represented by SEQ ID NO: 2 between breast cancer patients and a non-patient control group in Example 1 of the present disclosure.

### BEST MODE

The present disclosure is directed to a cancer diagnostic marker comprising at least one selected from the group consisting of S100A8, S100A9, ANXA2, KRT19, TRX1, GSN, APOC1, CA1, CHL1, FN1, LPA, MUC1 and PLAC1.

### MODE FOR INVENTION

According to one embodiment of the present disclosure, the present disclosure is directed to a cancer diagnostic marker comprising at least one selected from the group consisting of S100A8, S100A9, ANXA2, KRT19, TRX1, GSN, APOC1, CA1, CHL1, FN1, LPA, MUC1 and PLAC1.

In the present disclosure, the term “cancer” as a disease to be diagnosed represents or refers to a physiological condition characterized by atypical and uncontrolled cell growth in mammals. In the present disclosure, the cancer to be diagnosed may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma. Preferably, the cancer may be breast cancer.

In the present disclosure, the “S100A8” is 5100 calcium-binding protein A8 (S100A8), which corresponds to a protein encoded by the S100A8 gene in humans. In addition, the S100A8 is also called calgranulin A. S100A8 and S100A9 form a heterodimer called calprotectin. The protein encoded by the S100A8 gene is a member of the 5100 family of proteins containing 2 EF-hand calcium-binding motifs. The 5100 proteins are localized in the cytoplasm and/or nucleus in a wide range of cells and involved in a number of cellular processes such as cell cycle progression and differentiation. The 5100 genes include at least 13 members located as a cluster on chromosome 1q21, and this protein functions to inhibit casein kinase or as a cytokine. Changes in the expression of this protein are known to be associated with cystic fibrosis disease. In the present disclosure, the S100A8 protein may consist of the amino acid sequence represented by SEQ ID NO: 1, but is not limited thereto.

In the present disclosure, the “S100A9” is 5100 calcium-binding protein A9, which corresponds to a protein encoded by the S100A9 gene in humans. In addition, the S100A9 is also called migration inhibitory factor-related protein 14 (MRP14) or calgranulin B. S100A8 and S100A9 form a heterodimer called calprotectin. The S100A9 protein is also a member of the 5100 family of proteins containing 2 EF-hand calcium-binding motifs. In the present disclosure, the S100A9 protein may consist of the amino acid sequence represented by SEQ ID NO: 2, but is not limited thereto.

In the present disclosure, the “ANXA2” is an annexin A2 protein encoded by the ANXA2 gene, and is also called annexin II. ANXA2 is involved in a variety of cellular processes, such as cell motility (especially that of epithelial cells), linkage of membrane-associated protein complexes to the actin cytoskeleton, endocytosis, fibrinolysis, ion channel formation, and cell matrix interactions. In addition, the ANXA2 is a calcium-dependent phospholipid-binding protein whose function is to help organize exocytosis of intracellular proteins to the extracellular domain. In the present disclosure, the ANXA2 may consist of the amino acid sequence represented by SEQ ID NO: 3, but is not limited thereto.

In the present disclosure, the “KRT19” is a keratin type I cytoskeleton 19 encoded by the KRT19 gene, and is also known as cytokeratin-19 (CK-19) or keratin-19 (K19). KRT19 is 40 kDa in size and is a member of the keratin family. The keratins are intermediate filament proteins responsible for the structural integrity of epithelial cells and may be subdivided into cytokeratins and hair keratins. In the present disclosure, the KRT19 may consist of the amino acid sequence represented by SEQ ID NO: 4, but is not limited thereto.

In the present disclosure, the “TRX1” is also called thioredoxin-1 (Trx1) or TXN, and acts as a very important signaling molecule not only in response to redox perturbations, but also in cellular growth, regulation of gene expression, and apoptosis. The active site of the TRX1 corresponds to a conserved C-G-P-C motif. In the present disclosure, the TRX1 may consist of the amino acid sequence represented by SEQ ID NO: 5, but is not limited thereto.

In the present disclosure, the “GSN” is called gelsolin and is an actin-binding protein that is a key regulator of actin filament assembly and disassembly. The GSN is a member of the actin-severing gelsolin/villin superfamily, as it severs with nearly 100% efficiency. Gelsolin is found intracellularly (cytosol and mitochondria) and extracellularly (plasma). In the present disclosure, the GSN may consist of the amino acid sequence represented by SEQ ID NO: 6, but is not limited thereto.

In the present disclosure, the “APOC1” is apolipoprotein Cl, a protein encoded by a gene belonging to the apolipoprotein C family. It is highly expressed mainly in the liver and is activated when monocytes differentiate into macrophages. In the present disclosure, the APOC1 may consist of the amino acid sequence represented by SEQ ID NO: 7, but is not limited thereto.

In the present disclosure, the “CA1” is also called carbonic anhydrase 1, and is an enzyme encoded by the CA1 gene. Carbonic anhydrases are a large family of zinc metalloenzymes that catalyze the reversible hydration of carbon dioxide. They are known to participate in a variety of biological processes, including cellular respiration, calcification, acid-base balance, bone resorption, and the formation of cerebrospinal fluid, saliva, and gastric acid. In the present disclosure, the CA1 may consist of the amino acid sequence represented by SEQ ID NO: 8, but is not limited thereto.

In the present disclosure, the “CHL1” is called a neural cell adhesion molecule L1-like protein or a close homolog of L1, and is a protein encoded by the CHL1 gene. CHL1 is a cell adhesion molecule very closely related to L1, and CHL1 gene expression in melanocytes is regulated by MITF. The CHL1 also acts as helicase in the interphase of mitosis. In the present disclosure, the CHL1 may consist of the amino acid sequence represented by SEQ ID NO: 9, but is not limited thereto.

In the present disclosure, the “FN1” is fibronectin 1 which is a glycoprotein existing as a soluble dimer in plasma and as a dimer or multimer on cell surfaces or in extracellular matrices. This fibronectin protein is involved in cell adhesion and migration processes including embryogenesis, wound healing, blood coagulation, host defense, and metastasis. In the present disclosure, the FN1 may consist of the amino acid sequence represented by SEQ ID NO: 10, but is not limited thereto.

In the present disclosure, the “LPA” is called lipoprotein (a) or Lp (a), and is known as a risk factor for atherosclerotic diseases such as coronary heart disease or stroke. In the present disclosure, the LPA may consist of the amino acid sequence represented by SEQ ID NO: 11, but is not limited thereto.

In the present disclosure, the “MUC1” is a protein encoded by the MUC1 gene, and is a glycoprotein with extensive O-linked glycosylation of its extracellular domain. Mucins line the apical surface of epithelial cells in the lungs, stomach, intestines, eyes and several other organs. Mucins protect the body from infection by pathogen binding to oligosaccharides in the extracellular domain. In the present disclosure, the MUC1 may consist of the amino acid sequence represented by SEQ ID NO: 12, but is not limited thereto.

In the present disclosure, the “PLAC1” is a placenta-specific 1 (Placenta-specific 1), which is an X-linked trophoblast expressed at high levels in the placenta. In the present disclosure, PLAC1 may consist of the amino acid sequence represented by SEQ ID NO: 13, but is not limited thereto.

According to another embodiment of the present disclosure, the present disclosure is directed to a cancer diagnostic composition containing an agent for measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein.

In the present disclosure, the cancer to be diagnosed may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma. Preferably, the cancer may be breast cancer.

In the present disclosure, the “diagnosis” or “diagnosing” includes: determining the susceptibility of a subject to a specific disease or disorder; determining whether or not a subject currently has a particular disease or disorder; determining the prognosis of a subject with a specific disease or disorder (e.g., identification of pre-metastatic or metastatic cancer conditions, determination of cancer stages, or determination of responsiveness of cancer to therapy); or therametrics (e.g., monitoring states of a subject to provide information about treatment effects). With regard to the purposes of the present disclosure, the diagnosis or diagnosing refers to determining whether or not the above-described cancer has developed or the likelihood (risk) of developing the cancer.

In the present disclosure, the agent for measuring the expression level of the S100A8, S100A9, ANXA2, KRT19, TRX1, GSN, APOC1, CA1, CHL1, FN1, LPA, MUC1 or PLAC1 protein is not particularly limited, but may comprise, for example, at least one selected from the group consisting of an antibody, an oligopeptide, a ligand, a peptide nucleic acid (PNA) and an aptamer, which bind specifically to the S100A8, S100A9, ANXA2, KRT19, TRX1, GSN, APOC1, CA1, CHL1, FN1, LPA, MUC1 or PLAC1 protein.

In the present disclosure, the “antibody” refers to a substance that binds specifically to an antigen, causing an antigen-antibody reaction. With regard to the purposes of the present disclosure, the antibody refers to an antibody that binds specifically to the S100A8, S100A9, ANXA2, KRT19, TRX1, GSN, APOC1, CA1, CHL1, FN1, LPA, MUC1 or PLAC1 protein. The antibodies of the present disclosure include all polyclonal antibodies, monoclonal antibodies, and recombinant antibodies. The antibody may be easily produced using techniques well known in the art. For example, the polyclonal antibody may be produced by a method well known in the art, which comprises a process of injecting the protein antigen into an animal, collecting blood from the animal, and isolating serum containing the antibody. This polyclonal antibody may be produced from any animal species such as goats, rabbits, sheep, monkeys, horses, pigs, cattle, or dogs. In addition, the monoclonal antibody may be produced using a hybridoma method (see Kohler and Milstein (1976) European Journal of Immunology 6:511-519) well known in the art, or phage antibody library technology (see Clackson et al, Nature, 352:624-628, 1991; Marks et al, J. Mol. Biol., 222:58, 1-597, 1991). The antibody produced by the above method may be isolated and purified using methods such as gel electrophoresis, dialysis, salt precipitation, ion exchange chromatography, and affinity chromatography. In addition, the antibodies of the present disclosure include functional fragments of antibody molecules as well as complete forms having two full-length light chains and two full-length heavy chains. The expression “functional fragments of antibody molecules” refers to fragments retaining at least an antigen-binding function, and examples of the functional fragments include Fab, F(ab′), F(ab′)2, and Fv.

In the present disclosure, “peptide nucleic acid (PNA)” refers to an artificially synthesized polymer similar to DNA or RNA, and was first introduced by professors Nielsen, Egholm, Berg and Buchardt (at the University of Copenhagen, Denmark) in 1991. DNA has a phosphate-ribose backbone, whereas PNA has a backbone composed of repeating units of N-(2-aminoethyl)-glycine linked by peptide bonds. Thanks to this structure, PNA has a significantly increased binding affinity for DNA or RNA and a significantly increased stability, and thus is used in molecular biology, diagnostic analysis, and antisense therapy. PNA is disclosed in detail in Nielsen P E, Egholm M, Berg R H, Buchardt O (December 1991). “Sequence-selective recognition of DNA by strand displacement with a thymine-substituted polyamide”. Science 254 (5037): 1497-1500.

In the present disclosure, the “aptamer” is an oligonucleic acid or peptide molecule, and general contents of the aptamer are disclosed in detail in Bock L C et al., Nature 355(6360):5646(1992); Hoppe-Seyler F, Butz K “Peptide aptamers: powerful new tools for molecular medicine”. J Mol Med. 78(8):42630(2000); Cohen B A, Colas P, Brent R. “An artificial cell-cycle inhibitor isolated from a combinatorial library”. Proc Natl Acad Sci USA. 95(24): 142727(1998).

In the present disclosure, the agent for measuring the expression level of the gene encoding the S100A8, S100A9, ANXA2, KRT19, TRX1 GSN, APOC1, CA1, CHL1, FN1, LPA, MUC1 or PLAC1 protein may comprise at least one selected from the group consisting of a primer, a probe and an antisense nucleotide, which bind specifically to the gene encoding the S100A8, S100A9, ANXA2, KRT19, TRX1, GSN, APOC1, CA1, CHL1, FN1, LPA, MUC1 or PLAC1 protein.

In the present disclosure, the “primer” is a fragment that recognizes a target gene sequence, and includes a pair of forward and reverse primers. Preferably, the primer is a primer pair that provides analysis results with specificity and sensitivity. Because the nucleotide sequence of the primer does not match a non-targeted sequence in a sample, the primer can show high specificity when it amplifies only a target gene sequence containing a complementary primer binding site without causing non-specific amplification.

In the present disclosure, the “probe” refers to a substance which is capable of binding specifically to the target substance to be detected in a sample and may specifically identify the presence of the target substance in the sample through the binding. The kind of the probe is not specifically limited, as long as it is a substance that is generally used in the art. Preferably, the probe may be PNA (peptide nucleic acid), LNA (locked nucleic acid), a peptide, a polypeptide, a protein, RNA or DNA. More preferably, the probe is PNA. More specifically, the probe may be a biomaterial derived from an organism, an analogue thereof, or a material produced ex vivo, and examples thereof include enzymes, proteins, antibodies, microorganisms, animal/plant cells and organs, neural cells, DNA, and RNA. Examples of the DNA include cDNA, genomic DNA, and oligonucleotides, examples of the RNA include genomic RNA, mRNA, and oligonucleotides, and examples of the protein include antibodies, antigens, enzymes, and peptides.

In the present disclosure, the “LNA (locked nucleic acid” refers to a nucleic acid analog containing a 2′-O or 4′-C methylene bridge [J Weiler, J Hunziker and J Hall Gene Therapy (2006) 13, 496.502]. LNA nucleosides include common nucleic acid bases of DNA and RNA, and can form base pairs according to the Watson-Crick base pairing rule. However, due to ‘locking’ of the molecule attributable to the methylene bridge, the LNA fails to form an ideal shape in the Watson-Crick bond. When the LNA is incorporated in a DNA or RNA oligonucleotide, it can more rapidly pair with a complementary nucleotide chain, thus increasing the stability of the double strand. In the present disclosure, the “antisense” refers to an oligomer having a sequence of nucleotide bases and a subunit-to-subunit backbone that allows the antisense oligomer to hybridize to a target sequence in an RNA by Watson-Crick base pairing, to form an RNA:oligomer heteroduplex within the target sequence, typically with an mRNA. The oligomer may have exact sequence complementarity to the target sequence or near complementarity.

Information on either the S100A8, S100A9, ANXA2, KRT19, TRX1, GSN, APOC1, CA1, CHL1, FN1, LPA, MUC1 or PLAC1 protein according to the present disclosure, or the gene encoding this protein, is known. Based on this information, those skilled in the art can easily design a primer, a probe or an antisense nucleotide, which specifically binds to the gene encoding the protein.

According to still another embodiment of the present disclosure, the present disclosure is directed to a cancer diagnostic kit comprising the cancer diagnostic composition according to the present disclosure.

In the present disclosure, it is possible to diagnose the development, likelihood of development, responsiveness to therapy, prognosis, stage, likelihood of recurrence, etc. of a cancer disease by using the diagnostic kit.

In the present disclosure, the cancer to be diagnosed may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma. Preferably, the cancer may be breast cancer.

In the present disclosure, the kit may be, but is not limited to, an RT-PCR kit, a DNA chip kit, an ELISA kit, a protein chip kit, a rapid kit or a multiple-reaction monitoring (MRM) kit.

The cancer diagnostic kit of cancer according to the present disclosure may further include one or more other component compositions, solutions or devices suitable for analysis methods.

For example, the cancer diagnostic kit according to the present disclosure may further comprise essential elements necessary for performing reverse transcription polymerase reaction. The reverse transcription polymerase reaction kit comprises a pair of primers specific to a gene encoding a marker protein. Each primer is a nucleotide having a sequence specific to the nucleic acid sequence of the gene, and may have a length of about 7 bp to 50 bp, more preferably about 10 bp to 30 bp. In addition, the kit may comprise primers specific to the nucleic acid sequence of a control gene. In addition, the reverse transcription polymerase reaction kit may comprise a test tube or other suitable container, buffers (having various pHs and magnesium concentrations), deoxynucleotides (dNTPs), enzymes such as Taq-polymerase and reverse transcriptase, DNAse and RNAse inhibitors, DEPC-water, sterile water, and the like.

In addition, the diagnostic kit of the present disclosure may comprise essential elements necessary for performing DNA chip assay. The DNA chip kit may comprise a substrate to which a gene or a cDNA or oligonucleotide corresponding to a fragment thereof is attached, and reagents, agents, and enzymes for constructing a fluorescently labeled probe. In addition, the substrate may comprise a control gene or a cDNA or oligonucleotide corresponding to a fragment thereof.

In addition, the diagnostic kit of the present disclosure may comprise essential elements necessary for performing ELISA. The ELISA kit may comprise an antibody specific to the protein. The antibody has high specificity and affinity for the marker protein, with no cross-reactivity to other proteins, and may be a monoclonal antibody, a polyclonal antibody, or a recombinant antibody. Furthermore, the ELISA kit may comprise an antibody specific to a control protein. In addition, the ELISA kit may further comprise reagents capable of detecting the bound antibody, for example, a labeled secondary antibody, chromophores, an enzyme (e.g., conjugated with the antibody) and a substrate thereof, or other substance capable of binding to the antibody.

In the diagnostic kit of the present disclosure, as a support for antigen-antibody binding reaction, there may be used a well plate synthesized from a nitrocellulose membrane, a PVDF membrane, a polyvinyl resin or a polystyrene resin, or a glass slide made of glass, without being limited thereto.

In the diagnostic kit of the present disclosure, a label for the secondary antibody is preferably a conventional chromogenic agent for color development, and examples of the label include, but are not limited to, fluoresceins such as HRP (horseradish peroxidase), alkaline phosphatase, colloid gold, FITC (poly L-lysine-fluorescein isothiocyanate), RITC (rhodamine-B-isothiocyanate), and dyes.

In the diagnostic kit of the present disclosure, a chromogenic substrate for inducing color development is preferably selected depending on the label for color development, and may be TMB (3,3′,5,5′-tetramethyl benzidine), ABTS [2,2′-azino-bis(3-ethylbenzothiazoline-6-sulfonic acid)], or OPD (o-phenylenediamine). At this time, the chromogenic substrate is more preferably provided as dissolved in buffer (0.1M NaOAc, pH 5.5). A chromogenic substrate such as TMB is degraded by HRP, used as a label for the secondary antibody conjugate, to form a chromogen, and the presence of the marker protein is detected by visually checking the degree of deposition of the chromogen.

The washing solution in the diagnostic kit of the present disclosure preferably comprises phosphate buffer, NaCl and Tween 20. More preferably, the washing solution is a buffer solution (PBST) consisting of 0.02 M phosphate buffer, 0.13 M NaCl, and 0.05% Tween 20. After the antigen-antibody binding reaction, the secondary antibody is allowed to react with the antigen-antibody complex, and then the support is washed 3 to 6 times with a suitable amount of the washing solution. As the reaction stop solution, a sulfuric acid solution is preferably used.

According to yet another embodiment of the present disclosure, the present disclosure is directed to a method for providing information for cancer diagnosis, the method comprising a step of measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in a biological sample isolated from a subject of interest.

In the present disclosure, the “subject of interest” refers to a subject in whom whether or not the cancer has developed is uncertain and who has a high likelihood of developing the cancer.

In the present disclosure, the “biological sample” refers to any material, biological fluid, tissue or cells obtained or derived from the subject. For example, the biological sample may include whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid. Preferably, the biological sample may be a liquid biopsy (e.g., patient's tissue, cells, blood, serum, plasma, saliva, sputum or ascites, etc.) collected for histopathological examination by inserting a hollow needle or the like into an in vivo organ without incision of the skin of a patient having a high likelihood of developing cancer.

The method of the present disclosure may comprise a step of measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in a biological sample isolated as described above.

In the present disclosure, an agent for measuring the expression level of the S100A8, S100A9, ANXA2, KRT19, TRX1 GSN, APOC1, CA1, CHL1, FN1, LPA, MUC1 or PLAC1 protein is not particularly limited, but preferably may comprise at least one selected from the group consisting of an antibody, an oligopeptide, a ligand, a peptide nucleic acid (PNA) and an aptamer, which bind specifically to the S100A8, S100A9, ANXA2, KRT19, TRX1, GSN, APOC1, CA1, CHL1, FN1, LPA, MUC1 or PLAC1 protein.

In the present disclosure, methods for measurement or comparative analysis of the expression level of the S100A8, S100A9, ANXA2, KRT19, TRX1, GSN, APOC1, CA1, CHL1, FN1, LPA, MUC1 or PLAC1 protein include, but are not limited to, protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoelectrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, or ELISA (enzyme-linked immunosorbent assay).

In the present disclosure, a method for measurement or comparative analysis of the expression level of the S100A8, S100A9, ANXA2, KRT19, TRX1, GSN, APOC1, CA1, CHL1, FN1, LPA, MUC1 or PLAC1 protein may be performed by a multiple-reaction monitoring (MRM) method.

In the present disclosure, the multiple-reaction monitoring method may be performed using mass-spectrometry, preferably triple-quadrupole mass spectrometry.

In the present disclosure, the multiple-reaction monitoring (MRM) method using mass-spectrometry is an analysis technique capable of monitoring a change in concentration of a specific analyte by selectively isolating, detecting and quantifying the specific analyte. MRM is a method that can quantitatively and accurately measure multiple substances such as trace amounts of biomarkers present in a biological sample. In MRM, mother ions among the ion fragments generated in an ionization source are selectively transmitted to a collision tube by a first mass filter Q1. Then, the mother ions arriving at the collision tube collide with an internal collision gas, are fragmented to generate daughter ions which are then sent to a second mass filter Q2, where only characteristic ions are transmitted to a detection unit. MRM is an analysis method with high selectivity and sensitivity that can detect only information on a component of interest. MRM is used for quantitative analysis of small molecules and is used to diagnose specific genetic diseases. The MRM method has advantages in that it is easy to simultaneously measure multiple peptides, and it is possible to confirm the relative concentration difference of protein diagnostic marker candidates between a normal person and a cancer patient without using an antibody. In addition, since the MRM analysis method has excellent sensitivity and selectivity, it has been introduced for the analysis of complex proteins and peptides in blood, particularly in proteomic analysis using a mass spectrometer (Anderson L. et al., Mol CellProteomics, 5: 375-88, 2006; DeSouza, L. V. et al., Anal. Chem., 81: 3462-70, 2009).

In the present disclosure, to analyze the expression level of the S100A8, S100A9, ANXA2, KRT19, TRX1, GSN, APOC1, CA1, CHL1, FN1, LPA, MUC1 or PLAC1 protein by the multiple-reaction monitoring method, a target peptide capable of representing the S100A8, S100A9, ANXA2, KRT19, TRX1, GSN, APOC1, CA1, CHL1, FN1, LPA, MUC1 or PLAC1 protein may be selected.

In addition, in the present disclosure, to analyze the expression level of the S100A8, S100A9, ANXA2, KRT19, TRX1, GSN, APOC1, CA1, CHL1, FN1, LPA, MUC1 or PLAC1 protein by the multiple-reaction monitoring method, a pair of mother and daughter ions in the selected target peptide may be selected.

In the present disclosure, Table 1 below shows information about the target peptide representing the S100A8, S100A9, ANXA2, KRT19, TRX1, GSN, APOC1, CA1, CHL1, FN1, LPA, MUC1 or PLAC1 protein and a pair of mother and daughter ions of the target peptide.

TABLE 1

| | Gene | | | | | | Daughter |
|---|---|---|---|---|---|---|---|
| | name | Protein name | uniprotKB | Peptide sequence | | Mother ion | ion |
| 1 | S100A8 | Protein S100-A8 | P05109 | 1 | MLTELEK | 432.23 | 732.41 |
| | | | | | (SEQ ID NO: 14) | 432.23 | 619.33 |
| | | | | | | 432.23 | 518.28 |
| | | | | 2 | ALNSIIDVYHK | 636.85 | 887.50 |
| | | | | | (SEQ ID NO: 15) | 636.85 | 774.41 |
| | | | | | | 636.85 | 661.33 |
| | | | | | | 636.85 | 546.30 |
| 2 | 5100A9 | Protein S100-A9 | P06702 | 1 | DLQNFLK | 439.24 | 649.37 |
| | | | | | (SEQ ID NO: 16) | 439.24 | 521.31 |
| | | | | | | 439.24 | 407.27 |
| | | | | 2 | LTWASHEK | 486.25 | 757.36 |
| | | | | | (SEQ ID NO: 17) | 486.25 | 571.28 |
| | | | | | | 486.25 | 500.25 |
| | | | | | | 486.25 | 413.21 |
| | | | | 3 | NIETIINTFHQYSVK | 602.98 | 908.46 |
| | | | | | (SEQ ID NO: 18) | 602.98 | 761.39 |
| | | | | | | 602.98 | 624.34 |
| | | | | | | 602.98 | 486.28 |
| 3 | ANXA2 | Annexin A2 | P07355 | 1 | DLYDAGVK | 440.72 | 652.33 |
| | | | | | (SEQ ID NO: 19) | 440.72 | 489.27 |
| | | | | | | 440.72 | 374.24 |
| | | | | | | 440.72 | 303.20 |
| | | | | 2 | QDIAFAYQR | 556.28 | 868.47 |
| | | | | | (SEQ ID NO: 20) | 556.28 | 755.38 |
| | | | | | | 556.28 | 684.35 |
| | | | | | | 556.28 | 537.28 |
| | | | | | | 556.28 | 466.24 |
| 4 | KRT19 | Keratin, type I | P08727 | 1 | DYSHYYTTIQDLR | 558.93 | 846.47 |
| | | cytoskeletal 19 | | | (SEQ ID NO: 21) | 558.93 | 745.42 |
| | | | | | | 558.93 | 644.37 |
| | | | | | | 558.93 | 531.29 |
| | | | | 2 | AALEDTLAETEAR | 695.35 | 789.41 |
| | | | | | (SEQ ID NO: 22) | 695.35 | 676.33 |
| | | | | | | 695.35 | 605.29 |
| | | | | | | 695.35 | 476.25 |
| | | | | | | 695.35 | 375.20 |
| 5 | TRX1 | Thioredoxin | P10599 | 1 | TAFQEALDAAGDK | 668.82 | 889.43 |
| | | | | | (SEQ ID NO: 23) | 668.82 | 760.38 |
| | | | | | | 668.82 | 689.35 |
| | | | | | | 668.82 | 576.26 |
| | | | | | | 668.82 | 461.24 |
| | | | | 2 | CMPTFQFFK | 603.28 | 914.48 |
| | | | | | (SEQ ID NO: 24) | 603.28 | 817.42 |
| | | | | | | 603.28 | 716.38 |
| | | | | | | 603.28 | 569.31 |
| | | | | | | 603.28 | 441.25 |
| | | | | 3 | VGEFSGANK | 454.727 | 809.379 |
| | | | | | (SEQ ID NO: 49) | 454.727 | 752.357 |
| | | | | | | 454.727 | 623.315 |
| | | | | | | 454.727 | 476.246 |
| | | | | | | 454.727 | 389.214 |
| | | | | 4 | TAFQEALDAAGDK | 668.823 | 889.426 |
| | | | | | (SEQ ID NO: 50) | 668.823 | 760.384 |
| | | | | | | 668.823 | 689.346 |
| | | | | | | 668.823 | 576.262 |
| 6 | GSN | Gelsolin | P06396 | 1 | TGAQELLR | 444.25 | 729.43 |
| | | | | | (SEQ ID NO: 25) | 444.25 | 658.39 |
| | | | | | | 444.25 | 530.33 |
| | | | | | | 444.25 | 401.29 |
| | | | | 2 | YIETDPANR | 539.76 | 802.37 |
| | | | | | (SEQ ID NO: 26) | 539.76 | 673.33 |
| | | | | | | 539.76 | 572.28 |
| | | | | | | 539.76 | 457.25 |
| | | | | | | 539.76 | 360.20 |
| 7 | APOC1 | Apolipoprotein | P02654 | 1 | TPDVSSALDK | 516.76 | 719.39 |
| | | C-I | | | (SEQ ID NO: 27) | 516.76 | 620.32 |
| | | | | | | 516.76 | 533.29 |
| | | | | | | 516.76 | 446.26 |
| | | | | 2 | EFGNTLEDK | 526.75 | 605.31 |
| | | | | | (SEQ ID NO: 28) | 526.75 | 776.38 |
| | | | | | | 526.75 | 719.36 |
| | | | | | | 526.75 | 504.27 |
| | | | | | | 526.75 | 391.18 |
| | | | | 3 | EWFSETFQK | 601.28 | 886.43 |
| | | | | | (SEQ ID NO: 29) | 601.28 | 739.36 |
| | | | | | | 601.28 | 652.33 |
| | | | | | | 601.28 | 523.29 |
| | | | | 4 | QSELSAK | 601.28 | 422.24 |
| | | | | | (SEQ ID NO: 30) | 381.70 | 547.31 |
| | | | | | | 381.70 | 418.27 |
| | | | | | | 381.70 | 305.18 |
| 8 | CA1 | Carbonic | P00915 | 1 | VLDALQAIK | 485.80 | 758.44 |
| | | anhydrase 1 | | | (SEQ ID NO: 31) | 485.80 | 643.41 |
| | | | | | | 485.80 | 572.38 |
| | | | | | | 485.80 | 459.29 |
| | | | | 2 | ADGLAVIGVLMK | 593.85 | 759.48 |
| | | | | | (SEQ ID NO: 32) | 593.85 | 660.41 |
| | | | | | | 593.85 | 547.33 |
| | | | | | | 593.85 | 490.31 |
| 9 | CHL1 | Neural cell | O00533 | 1 | IIPSNNSGTFR | 603.32 | 490.23 |
| | | adhesion | | | (SEQ ID NO: 33) | 603.32 | 759.37 |
| | | molecule L1-like | | | | 603.32 | 681.33 |
| | | protein | | | | 603.32 | 567.29 |
| | | | | | | 603.32 | 480.26 |
| | | | | 2 | VIAVNEVGR | 478.78 | 744.40 |
| | | | | | (SEQ ID NO: 34) | 478.78 | 673.36 |
| | | | | | | 478.78 | 574.29 |
| | | | | | | 478.78 | 460.25 |
| | | | | 3 | GDLYFANVEEK | 642.81 | 836.42 |
| | | | | | (SEQ ID NO: 35) | 642.81 | 689.35 |
| | | | | | | 642.81 | 618.31 |
| | | | | | | 642.81 | 504.27 |
| | | | | 4 | IENVSYQDK | 548.27 | 853.41 |
| | | | | | (SEQ ID NO: 36) | 548.27 | 739.36 |
| | | | | | | 548.27 | 640.29 |
| | | | | | | 548.27 | 553.26 |
| | | | | | | 548.27 | 390.20 |
| | | | | 5 | YHIYENGTLQINR | 540.94 | 915.50 |
| | | | | | (SEQ ID NO: 37) | 540.94 | 801.46 |
| | | | | | | 540.94 | 744.44 |
| | | | | | | 540.94 | 643.39 |
| | | | | | | 540.94 | 530.30 |
| 10 | FN1 | Fibronectin | P02751 | 1 | SYTITGLQPGTDYK | 772.39 | 808.38 |
| | | | | | (SEQ ID NO: 38) | 772.39 | 680.32 |
| | | | | | | 772.39 | 583.27 |
| | | | | | | 772.39 | 526.25 |
| | | | | 2 | TYHVGEQWQK | 425.88 | 1011.50 |
| | | | | | (SEQ ID NO: 39) | 425.88 | 874.44 |
| | | | | | | 425.88 | 775.37 |
| | | | | | | 425.88 | 718.35 |
| | | | | 3 | HEEGHMLNCTCFGQ | 644.94 | 985.40 |
| | | | | | GR | 644.94 | 825.37 |
| | | | | | (SEQ ID NO: 40) | 644.94 | 724.32 |
| | | | | | | 644.94 | 564.29 |
| | | | | | | 644.94 | 417.22 |
| | | | | 4 | STTPDITGYR | 555.78 | 922.46 |
| | | | | | (SEQ ID NO: 41) | 555.78 | 821.42 |
| | | | | | | 555.78 | 724.36 |
| | | | | | | 555.78 | 609.34 |
| 11 | LPA | Apolipoprotein | P08519 | 1 | WVLTAAHCLK | 400.22 | 400.71 |
| | | (a) | | | (SEQ ID NO: 42) | 400.22 | 800.41 |
| | | | | | | 400.22 | 699.36 |
| | | | | | | 400.22 | 628.32 |
| | | | | | | 400.22 | 557.29 |
| | | | | 2 | GTYSTTVTGR | 521.76 | 634.30 |
| | | | | | (SEQ ID NO: 43) | 521.76 | 884.45 |
| | | | | | | 521.76 | 721.38 |
| | | | | | | 521.76 | 533.30 |
| | | | | 3 | YICAEHLAR | 566.78 | 696.38 |
| | | | | | (SEQ ID NO: 44) | 566.78 | 625.34 |
| | | | | | | 566.78 | 496.30 |
| | | | | | | 566.78 | 359.24 |
| | | | | 4 | NPDAVAAPYCYTR | 749.34 | 1171.56 |
| | | | | | (SEQ ID NO: 45) | 749.34 | 1100.52 |
| | | | | | | 749.34 | 1001.45 |
| | | | | | | 749.34 | 930.41 |
| 12 | MUC1 | Mucin-1 | P15941 | 1 | YVPPSSTDR | 511.25 | 759.36 |
| | | | | | (SEQ ID NO: 46) | 511.25 | 662.31 |
| | | | | | | 511.25 | 565.26 |
| | | | | | | 511.25 | 478.23 |
| | | | | | | 511.25 | 391.19 |
| 13 | PLAC1 | Placenta-specific | Q9HBJ0 | 1 | FVIPVSCAAPQK | 658.86 | 1070.57 |
| | | protein 1 | | | (SEQ ID NO: 47) | 658.86 | 957.48 |
| | | | | | | 658.86 | 860.43 |
| | | | | | | 658.86 | 761.36 |
| | | | | | | 658.86 | 674.33 |
| | | | | | | 658.86 | 514.30 |

In the present disclosure, in order to detect the S100A8, S100A9, ANXA2, KRT19, TRX1, GSN, APOC1, CA1, CHL1, FN1, LPA, MUC1 or PLAC1 protein, a peptide is synthesized by substituting some amino acids of the target peptide of each of the proteins with a stable isotope. When the synthesized peptide is used as an internal standard substance in multiple-reaction monitoring analysis, the absolute amount of the protein in blood may also be measured, thus further increasing the accuracy of the analysis.

In the present disclosure, as the internal standard substance, any internal standard substance that is generally used in the multiple-reaction monitoring analysis may be used. For example, E. coli beta-galactosidase may be used. The target peptide representing the E. coli beta-galactosidase may consist of the amino acid sequence of SEQ ID NO: 48, and mother and daughter ions thereof may have mass-to-charge ratios of m/z 542.3 and m/z 636.3, respectively, but are not limited thereto.

In addition, in the present disclosure, in order to measure the absolute amount of the S100A8, S100A9, ANXA2, KRT19, TRX1, GSN, APOC1, CA1, CHL1, FN1, LPA, MUC1 or PLAC1 protein in blood, a specific peptide synthesized by substituting some amino acids of the target peptide with a stable isotope is used as an internal standard substance. In this case, the amino acids substituted with the isotope are preferably lysine or arginine, but are not limited thereto. Here, as the synthesized peptide, an isolated peptide with a purity of 95% or higher is preferably used.

Meanwhile, in the present disclosure, an agent for measuring the expression level of the gene encoding the S100A8, S100A9, ANXA2, KRT19, TRX1 GSN, APOC1, CA1, CHL1, FN1, LPA, MUC1 or PLAC1 protein may comprise at least one selected from the group consisting of a primer, a probe and an antisense nucleotide, which bind specifically to the S100A8, S100A9, ANXA2, KRT19, TRX1, GSN, APOC1, CA1, CHL1, FN1, LPA, MUC1 or PLAC1 protein.

In the present disclosure, to measure the presence and expression level of the gene encoding the S100A8, S100A9, ANXA2, KRT19, TRX1, GSN, APOC1, CA1, CHL1, FN1, LPA, MUC1 or PLAC1 protein, an analysis method of measuring the mRNA level of the gene may be used, and examples thereof include, but are not limited to, reverse transcription-polymerase chain reaction (RT-PCR), competitive RT-PCR, real-time RT-PCR, RNase protection assay (RPA), Northern blotting, and DNA chip assay.

In one embodiment of the present disclosure, if the measured expression level of either at least one protein selected from the group consisting of S100A8, S100A9, ANXA2, KRT19, TRX1, GSN, APOC1, CA1, CHL1, FN1, LPA, MUC1 and PLAC1, or the gene encoding the protein, in the biological sample isolated from the subject of interest, increases or decreases compared to a normal control, it may be predicted that the subject has a high likelihood of developing the cancer.

In other embodiments of the present disclosure, it is possible to predict responsiveness to therapy, preferably responsiveness to anticancer chemotherapy or immunotherapy, by measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in a biological sample isolated from a subject of interest.

In another embodiment of the present disclosure, it is possible to predict the prognosis of a subject of interest, preferably the prognosis after surgical operation, by measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in a biological sample isolated from the subject of interest. Here, the subject of interest may be a subject who has had cancer and has undergone surgical resection.

In another embodiment of the present disclosure, it is possible to predict the stage of cancer in a subject of interest by measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in a biological sample isolated from the subject of interest.

In the present disclosure, the “stage” refers to the extent to which cancer cells have spread or the stage of cancer progression. The international classification according to the status of cancer progression generally follows the TNM stage classification. Here, ‘T (Tumor Size)’ is a classification according to the size of the primary tumor, ‘N (Lymph Node)’ is a classification according to the degree of lymph node metastasis, and ‘M (Metastasis)’ is a classification according to whether cancer has metastasized to other organs. Detailed classification for T, N and M is shown in Table 2 below, and the stage classification of cancer according to T, N and M is shown in Table 3 below.

TABLE 2

| TNM stage | | Definition |
|---|---|---|
| Size of the | T0 | Tumor cells that show the appearance of a malignant tumor, |
| primary tumor | | but are localized to the mucous membrane or epithelium of |
| (T stage) | | origin, and have not yet invaded the basement membrane. |
| | T1 | A lesion or tumor confined to the organ of origin, which is |
| | | mobile and has not invaded adjacent and surrounding tissues. |
| | T2 | A tumor with a size of about 2 to 5 cm. |
| | T3 | A tumor having a size larger than T2 but localized to the organ. |
| | T4 | Adhesion and infiltration into surrounding tissues. |
| Lymph node | N0 | There is no evidence of lymph node involvement. |
| status (N stage) | N1 | Invades one palpable and mobile lymph node (1 to 2 cm or |
| | | larger, usually up to 3 cm in size) limited to the first station. |
| | N2 | Palpable, partially mobile and firm or hard lymph nodes. |
| | | There is microscopic evidence of invasion, and involved |
| | | nodes are clinically entangled and show contralateral or |
| | | bilateral involvement (3 to 5 cm). |
| | N3 | Lymph nodes that are completely fixed, pass through the |
| | | capsule, are completely fixed to bones, large blood vessels, |
| | | skin, nerves, etc., and have a size of 6 cm or more. |
| Distant | M0 | There is no distant metastasis. |
| metastasis | M1 | There is distant metastasis. |
| (M stage) | | |

TABLE 3

| | | | | |
|---|---|---|---|---|
| Stage | | | | |
| classification | T1 | T2 | T3 | T4 |

| | | |
|---|---|---|
| N0 | Stage 1 | Stage 2 |

| | |
|---|---|
| N1 | Stage 3 |
| N2 | |
| N3 | |
| M1 | Stage 4 |

In another embodiment of the present disclosure, it is possible to predict the likelihood of recurrence of cancer by measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in a biological sample isolated from a subject of interest.

In the present disclosure, the cancer may be breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma. Preferably, the cancer may be breast cancer.

According to another embodiment of the present disclosure, the present disclosure is directed to a method for screening a drug for preventing or treating cancer, the method comprising steps of:

(a) treating either a sample isolated from a cancer subject or a cancer disease animal model with a candidate drug; and (b) measuring the expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in the sample or cancer disease animal model treated with the candidate drug.

In the present disclosure, the “sample” includes tissue, cells, whole blood, serum, blood plasma, tissue autopsy sample (e.g., brain, skin, lymph node, spinal cord, etc.), cell culture supernatant, disrupted eukaryotic cells and bacterial expression system, but is not limited. With regard to the purposes of the present disclosure, the isolated sample is preferably cells or tissue isolated from a cancer subject, but is not limited thereto. The biological sample may be allowed to react with the candidate drug for preventing or treating cancer in a manipulated or unmanipulated state.

In the present disclosure, the term “cancer disease animal model” refers to an animal other than humans, which is an animal that may be determined by a person skilled in the art to be in a pathological state of cancer. For example, the animal may be selected from the group consisting of rats, mice, guinea pigs, hamsters, rabbits, monkeys, dogs, cats, cattle, horses, pigs, sheep and goats, but is not limited thereto.

In the present disclosure, before the sample isolated from the cancer subject or the cancer disease animal mode is treated with the candidate drug, a step of measuring the expression level of at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in the sample or the cancer disease animal model, may be performed.

The term “candidate drug” in the present disclosure refers to a substance that may be applied to a cancer patient to alleviate or beneficially change the patient's symptoms caused by cancer. The candidate drug is a substance capable of reducing the expression or activity of either the S100A8, S100A9, ANXA2, KRT19, TRX1, GSN, APOC1, CA1, CHL1, FN1, LPA, MUC1 or PLAC1 protein, or a gene encoding the protein. Examples of the candidate drug include, but are not limited to, low-molecular-weight compounds, antibodies, antisense nucleotides, small interfering RNAs, short hairpin RNAs, nucleic acids, proteins, peptides, other extracts or natural products.

In the present disclosure, the method of measuring the expression level of either at least one protein selected from the group consisting of S100A8, S100A9, ANXA2, KRT19, TRX1, GSN, APOC1, CA1, CHL1, FN1, LPA, MUC1 and PLAC1, or the gene encoding the protein, in the sample or the cancer disease animal model before or after treatment with the candidate drug, and the agent used in the method, overlap with those described above with respect to the method for providing information for cancer diagnosis, and thus detailed description thereof will be omitted herein.

In the present disclosure, the method may further comprise step (c) of determining that the candidate drug is the drug for preventing or treating cancer, when the expression level of S100A8, S100A9, ANXA2, KRT19, TRX1, GSN, APOC1, CA1, CHL1, FN1, LPA, MUC1 or PLAC1 protein, or a gene encoding the protein, measured in step (b), decreases or increases compared to that before treatment with the candidate drug.

Hereinafter, the present disclosure will be described in detail with reference to examples. However, the following examples serve merely to illustrate the present disclosure, and the scope of the present disclosure is not limited by the following examples.

### EXAMPLES

### [Example 1] Detection of Biomarker Set for Diagnosis of Breast Cancer

1. Preparation of Samples

In order to evaluate the breast cancer diagnostic accuracy of a biomarker combination of the present disclosure, plasma was isolated from blood samples obtained from 66 breast cancer patients and 66 normal control persons, and total protein was quantified by Bradford assay. 200 μg of the total protein was modified with urea, and then reduced with dithiothreitol (DTT) and alkylated by iodoacetamide. Thereafter, trypsin was added at a ratio of 1:50 (w/w) based on the amount of the total protein to form a peptide, and salt was removed using a C18 column. As an internal standard substance, a synthetic product in which the amino acid group attached to the end of each peptide was substituted with an isotope was used.

2. Performing Multiple-Reaction Monitoring Using Triple-Quadrupole Mass Spectrometry

For triple quadrupole mass spectrometry, 13 target peptides of the biomarkers of the present disclosure and pairs of mother and daughter ions thereof were selected, and the results are shown in Table 1 above. The final samples prepared in Example 1.1 were subjected to reverse-phase resin chromatography to separate plasma peptide fragments, and MRM spectra of each peptide were obtained using triple quadrupole mass spectrometry (instrument: 5500 Qtrap, AB Sciex, USA). At this time, the reverse-phase resin chromatography was performed with HALO™ C18 column (Eksigent, USA) using a 5% to 40% acetonitrile concentration gradient for 45 minutes. Quantitative information was confirmed by calculating the peak area of the MRM chromatogram of each target peptide by MultiQuant™ computer quantitative analysis program (AB Sciex, USA). At this time, the quantitative value of each target peptide was expressed as a percentage relative to the peak area of the MRM chromatogram of E. coli beta-galactosidase added as an internal standard substance. The difference in the protein expression level between the breast cancer patients and the non-patient control patients could be determined by calculating the MRM chromatogram area ratio of each peptide.

3. Evaluation of Breast Cancer Diagnostic Accuracy of Each Combination of Biomarkers

The quantitative results of the 13 biomarkers identified in Example 1.2 were unified through logistic regression, and a single diagnostic marker consisting of a plurality of markers (multi-label marker) was prepared to evaluate the diagnostic efficiency of breast cancer thereof.

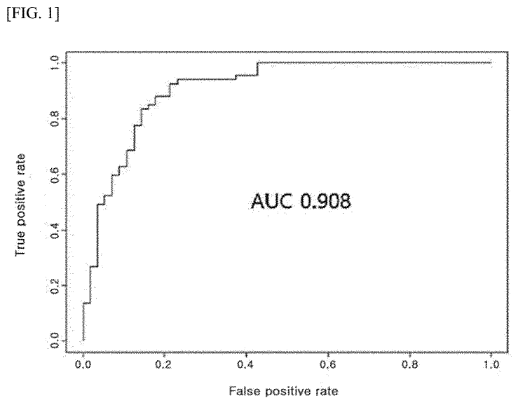

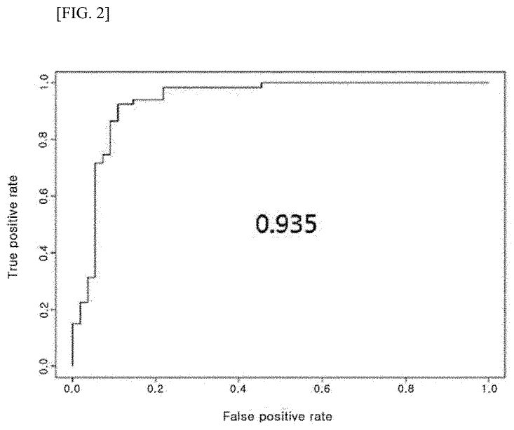

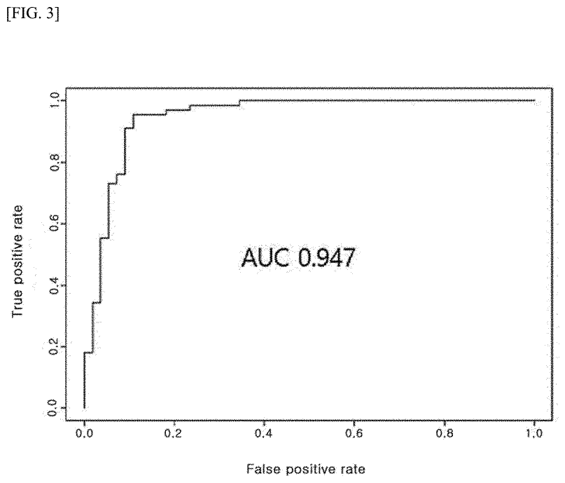

As a result, as shown in FIG. 1 showing a receiver operating characteristic (ROC) graph obtained for 66 breast cancer patients and 66 normal control persons using a combination of APOC1, CA1 and CHL1 among the 13 biomarkers of the present disclosure, the combination showed an AUC (area under the curve) of 0.908. In addition, as shown in FIG. 2, a combination of APOC1, CA1, CHL1, S100A9 and S100A8 biomarkers showed an AUC of 0.935. In addition, as shown in FIG. 3, a combination of APOC1, CA1, CHL1, S100A9, S100A8, ANXA2 and GSN biomarkers showed an AUC of 0.947.

Accordingly, it could be confirmed that the breast cancer diagnostic accuracy of the biomarker combination of the present disclosure was very high.

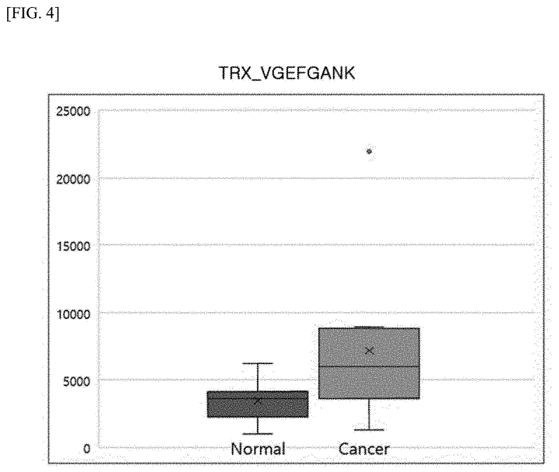

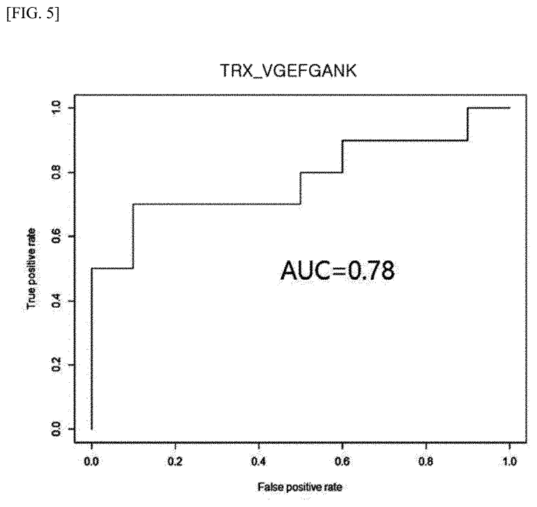

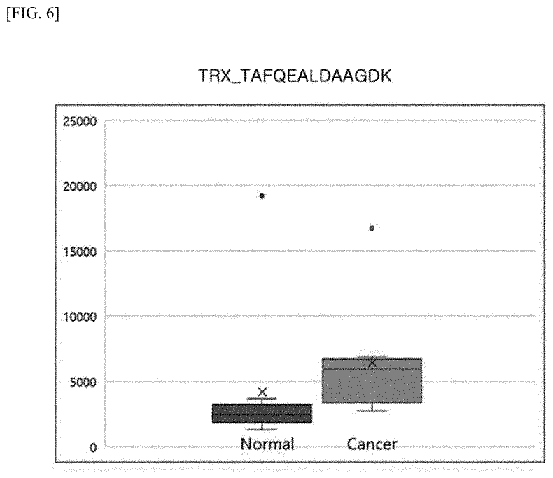

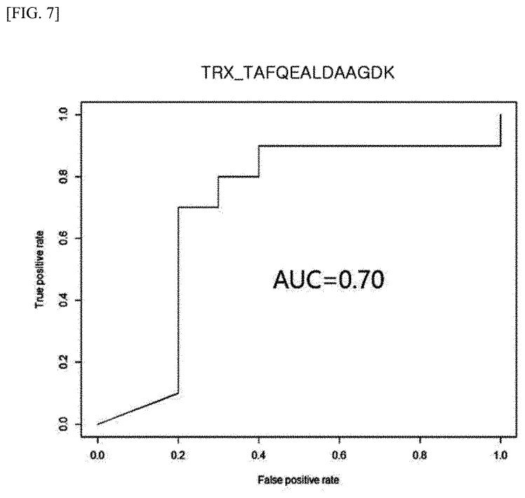

In addition, as shown in FIGS. 4, 5, 6 and 7 showing receiver operating characteristic (ROC) graphs obtained for 10 breast cancer patients and 10 normal control persons using the biomarkers of SEQ ID NOs: 49 and 50 among the TRX1 biomarkers of the present disclosure, the biomarkers showed an AUC (area under the curve) of 0.78, indicating that the biomarkers are single markers with a very high accuracy.

### INDUSTRIAL APPLICABILITY

The present disclosure relates to a composition capable of diagnosing cancer, a diagnostic kit comprising the same, and a method for providing information for diagnosing cancer using the composition.

1. A cancer diagnostic marker comprising at least one selected from the group consisting of S100A8, S100A9, ANXA2, KRT19, TRX1, GSN, APOC1, CA1, CHL1, FN1, LPA, MUC1 and PLAC1.

2. (canceled)

3. A cancer diagnostic composition containing an agent for measuring an expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein.

4. (canceled)

5. The cancer diagnostic composition of claim 3, wherein the agent for measuring the expression level of the S100A8, S100A9, ANXA2, KRT19, TRX1 GSN, APOC1, CA1, CHL1, FN1, LPA, MUC1 or PLAC1 protein comprises at least one selected from the group consisting of an antibody, an oligopeptide, a ligand, a peptide nucleic acid (PNA) and an aptamer, which bind specifically to the S100A8, S100A9, ANXA2, KRT19, TRX1, GSN, APOC1, CA1, CHL1, FN1, LPA, MUC1 or PLAC1 protein.

6. The cancer diagnostic composition of claim 3, wherein the agent for measuring the expression level of the gene encoding the S100A8, S100A9, ANXA2, KRT19, TRX1 GSN, APOC1, CA1, CHL1, FN1, LPA, MUC1 or PLAC1 protein comprises at least one selected from the group consisting of a primer, a probe and an antisense nucleotide, which bind specifically to the gene encoding the S100A8, S100A9, ANXA2, KRT19, TRX1, GSN, APOC1, CA1, CHL1, FN1, LPA, MUC1 or PLAC1 protein.

7-8. (canceled)

9. A method for providing information for cancer diagnosis, the method comprising a step of measuring an expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in a biological sample isolated from a subject of interest.

10. The method of claim 9, wherein the biological sample is whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid.

11. The method of claim 9, wherein an agent for measuring the expression level of the S100A8, S100A9, ANXA2, KRT19, TRX1 GSN, APOC1, CA1, CHL1, FN1, LPA, MUC1 or PLAC1 protein comprise at least one selected from the group consisting of an antibody, an oligopeptide, a ligand, a peptide nucleic acid (PNA) and an aptamer, which bind specifically to the S100A8, S100A9, ANXA2, KRT19, TRX1, GSN, APOC1, CA1, CHL1, FN1, LPA, MUC1 or PLAC1 protein.

12. The method of claim 9, wherein the measuring of the expression level of the S100A8, S100A9, ANXA2, KRT19, TRX1, GSN, APOC1, CA1, CHL1, FN1, LPA, MUC1 or PLAC1 protein is performed by protein chip assay, immunoassay, ligand binding assay, MALDI-TOF (Matrix Assisted Laser Desorption/Ionization Time of Flight Mass Spectrometry) assay, SELDI-TOF (Surface Enhanced Laser Desorption/Ionization Time of Flight Mass Spectrometry) assay, radioimmunoassay, radioimmunodiffusion, Ouchterlony immunodiffusion, rocket immunoelectrophoresis, immunohistochemical staining, complement fixation assay, two-dimensional electrophoresis assay, liquid chromatography-mass spectrometry (LC-MS), LC-MS/MS (liquid chromatography-mass spectrometry/mass spectrometry), Western blotting, or ELISA (enzyme-linked immunosorbent assay).

13. The method of claim 9, wherein the measuring of the expression level of the S100A8, S100A9, ANXA2, KRT19, TRX1, GSN, APOC1, CA1, CHL1, FN1, LPA, MUC1 or PLAC1 protein is performed by a multiple-reaction monitoring (MRM) method.

14. The method of claim 13, wherein a target peptide representing the S100A8 consists of the amino acid sequence represented by SEQ ID NO: 14 or 15; or
a target peptide representing the S100A9 consists of the amino acid sequence represented by any one of SEQ ID NOs: 16 to 18; or
a target peptide representing the ANXA2 consists of the amino acid sequence represented by SEQ ID NO: 19 or 20; or
a target peptide representing the KRT19 consists of the amino acid sequence represented by SEQ ID NO: 21 or 22; or
a target peptide representing the TRX1 consists of the amino acid sequence represented by SEQ ID NO: 23, 24, 49 or 50; or
a target peptide representing the GSN consists of the amino acid sequence represented by SEQ ID NO: 25 or 26; or
a target peptide representing the APOC1 consists of the amino acid sequence represented by any one of SEQ ID NOs: 27 to 30; or
a target peptide representing the CA1 consists of the amino acid sequence represented by SEQ ID NO: 31 or 32; or
a target peptide representing the CHL1 consists of the amino acid sequence represented by any one of SEQ ID NOs: 33 to 37; or
a target peptide representing the FN1 consists of the amino acid sequence represented by any one of SEQ ID NOs: 38 to 41; or
a target peptide representing the LPA consists of the amino acid sequence represented by any one of SEQ ID NOs: 42 to 45; or
a target peptide representing the MUC1 consists of the amino acid sequence represented by SEQ ID NO: 46; or
a target peptide representing the PLAC1 consists of the amino acid sequence represented by SEQ ID NO: 47.

15. The method of claim 13, wherein
a pair of mother and daughter ions of a target peptide representing the S100A8 have mass-to-charge ratios of 432.23 m/z and 732.41 m/z, 432.23 m/z and 619.33 m/z, 432.23 m/z and 518.28 m/z, 636.85 m/z and 887.50 m/z, 636.85 m/z and 774.41 m/z, 636.85 m/z and 661.33 m/z, or 636.85 m/z and 546.30 m/z, respectively; or
a pair of mother and daughter ions of a target peptide representing the S100A9 have mass-to-charge ratios of 439.24 m/z and 649.37 m/z, 439.24 m/z and 521.31 m/z, 439.24 m/z and 407.27 m/z, 486.25 m/z and 757.36 m/z, 486.25 m/z and 571.28 m/z, 486.25 m/z and 500.25 m/z, 486.25 m/z and 413.21 m/z, 602.98 m/z and 908.46 m/z, 602.98 m/z and 761.39 m/z, 602.98 m/z and 624.34 m/z, or 602.98 m/z and 486.28 m/z, respectively; or
a pair of mother and daughter ions of a target peptide representing the ANXA2 have mass-to-charge ratios of 440.72 m/z and 652.33 m/z, 440.72 m/z and 489.27 m/z, 440.72 m/z and 374.24 m/z, 440.72 m/z and 303.20 m/z, 556.28 m/z and 868.47 m/z, 556.28 m/z and 755.38 m/z, 556.28 m/z and 684.35 m/z, 556.28 m/z and 537.28 m/z, or 556.28 m/z and 466.24 m/z, respectively; or
a pair of mother and daughter ions of a target peptide representing the KRT19 have mass-to-charge ratios of 558.93 m/z and 846.47 m/z, 558.93 m/z and 745.42 m/z, 558.93 m/z and 644.37 m/z, 558.93 m/z and 531.29 m/z, 695.35 m/z and 789.41 m/z, 695.35 m/z and 676.33 m/z, 695.35 m/z and 605.29 m/z, 695.35 m/z and 476.25 m/z, or 695.35 m/z and 375.20 m/z, respectively; or
a pair of mother and daughter ions of a target peptide representing the TRX1 have mass-to-charge ratios of 668.82 m/z and 889.43 m/z, 668.82 m/z and 760.38 m/z, 668.82 m/z and 689.35 m/z, 668.82 m/z and 576.26 m/z, 668.82 m/z and 461.24 m/z, 603.28 m/z and 914.48 m/z, 603.28 m/z and 817.42 m/z, 603.28 m/z and 716.38 m/z, 603.28 m/z and 569.31 m/z, 603.28 m/z and 441.25 m/z, 454.727 m/z and 809.379 m/z, 454.727 m/z and 752.357 m/z, 454.727 m/z and 623.315 m/z, 454.727 m/z and 476.246 m/z, 454.727 m/z and 389.214 m/z, 668.823 m/z and 889.426 m/z, 668.823 m/z and 760.384 m/z, 668.823 m/z and 689.346 m/z, or 668.823 m/z and 576.262 m/z, respectively; or
a pair of mother and daughter ions of a target peptide representing the GSN have mass-to-charge ratios of 444.25 m/z and 729.43 m/z, 444.25 m/z and 658.39 m/z, 444.25 m/z and 530.33 m/z, 444.25 m/z and 401.29 m/z, 539.76 m/z and 802.37 m/z, 539.76 m/z and 673.33 m/z, 539.76 m/z and 572.28 m/z, 539.76 m/z and 457.25 m/z, or 539.76 m/z and 360.20 m/z, respectively; or
a pair of mother and daughter ions of a target peptide representing he APOC1 have mass-to-charge ratios of 516.76 m/z and 719.39 m/z, 516.76 m/z and 620.32 m/z, 516.76 m/z and 533.29 m/z, 516.76 m/z and 466.24 m/z, 526.75 m/z and 605.31 m/z, 526.75 m/z and 776.38 m/z, 526.75 m/z and 719.36 m/z, 526.75 m/z and 504.27 m/z, 526.75 m/z and 391.18 m/z, 601.28 m/z and 886.43 m/z, 601.28 m/z and 739.36 m/z, 601.28 m/z and 652.33 m/z, 601.28 m/z and 523.29 m/z, 601.28 m/z and 422.24 m/z, 381.70 m/z and 547.31 m/z, 381.70 m/z and 418.27 m/z, or 381.70 m/z and 305.18 m/z, respectively; or
a pair of mother and daughter ions of a target peptide representing the CA1 have mass-to-charge ratios of 485.80 m/z and 758.44 m/z, 485.80 m/z and 643.41 m/z, 485.80 m/z and 572.38 m/z, 485.80 m/z and 459.29 m/z, 593.85 m/z and 759.48 m/z, 593.85 m/z and 660.41 m/z, 593.85 m/z and 547.33 m/z, or 593.85 m/z and 490.31 m/z, respectively; or
a pair of mother and daughter ions of a target peptide representing the CHL1 have mass-to-charge ratios of 603.32 m/z and 490.23 m/z, 603.32 m/z and 795.37 m/z, 603.32 m/z and 681.33 m/z, 603.32 m/z and 567.29 m/z, 603.32 m/z and 480.26 m/z, 478.78 m/z and 744.40 m/z, 478.78 m/z and 673.36 m/z, 478.78 m/z and 574.29 m/z, 478.78 m/z and 460.25 m/z, 642.81 m/z and 836.42 m/z, 642.81 m/z and 689.35 m/z, 642.81 m/z and 618.31 m/z, 642.81 m/z and 504.27 m/z, 548.27 m/z and 853.41 m/z, 548.27 m/z and 739.36 m/z, 548.27 m/z and 640.29 m/z, 548.27 m/z and 553.26 m/z, 548.27 m/z and 390.20 m/z, 540.94 m/z and 915.50 m/z, 540.94 m/z and 801.46 m/z, 540.94 m/z and 744.44 m/z, 540.94 m/z and 643.39 m/z, or 540.94 m/z and 530.30 m/z, respectively; or
a pair of mother and daughter ions of a target peptide representing the FN1 have mass-to-charge ratios of 772.39 m/z and 808.38 m/z, 772.39 m/z and 680.32 m/z, 772.39 m/z and 583.27 m/z, 772.39 m/z and 526.25 m/z, 425.88 m/z and 1011.50 m/z, 425.88 m/z and 874.44 m/z, 425.88 m/z and 775.37 m/z, 425.88 m/z and 718.35 m/z, 644.94 m/z and 985.40 m/z, 644.94 m/z and 825.37 m/z, 644.94 m/z and 724.32 m/z, 644.94 m/z and 564.29 m/z, 644.94 m/z and 417.22 m/z, 555.78 m/z and 922.46 m/z, 555.78 m/z and 821.42 m/z, 555.78 m/z and 724.36 m/z, or 555.78 m/z and 609.34 m/z, respectively; or
a pair of mother and daughter ions of a target peptide representing the LPA have mass-to-charge ratios of 400.22 m/z and 400.71 m/z, 400.22 m/z and 800.41 m/z, 400.22 m/z and 699.36 m/z, 400.22 m/z and 628.32 m/z, 400.22 m/z and 557.29 m/z, 521.76 m/z and 634.30 m/z, 521.76 m/z and 884.45 m/z, 521.76 m/z and 721.38 m/z, 521.76 m/z and 533.30 m/z, 566.78 m/z and 696.38 m/z, 566.78 m/z and 625.34 m/z, 566.78 m/z and 496.30 m/z, 566.78 m/z and 359.24 m/z, 749.34 m/z and 1171.56 m/z, 749.34 m/z and 1100.52 m/z, 749.34 m/z and 1001.45 m/z, or 749.34 m/z and 930.41 m/z, respectively; or
a pair of mother and daughter ions of a target peptide representing the MUC1 have mass-to-charge ratios of 511.25 m/z and 759.36 m/z, 511.25 m/z and 662.31 m/z, 511.25 m/z and 565.26 m/z, 511.25 m/z and 478.23 m/z, or 511.25 m/z and 391.19 m/z, respectively; or
a pair of mother and daughter ions of a target peptide representing the PLAC1 have mass-to-charge ratios of 658.86 m/z and 1070.57 m/z, 658.86 m/z and 957.48 m/z, 658.86 m/z and 860.43 m/z, 658.86 m/z and 761.36 m/z, 658.86 m/z and 674.33 m/z, or 658.86 m/z and 514.30 m/z, respectively.

16. The method of claim 13, wherein an internal standard substance which is used in the multiple-reaction monitoring method is either a synthetic peptide obtained by substituting certain amino acids of a target peptide with an isotope, or E. coli beta-galactosidase.

17. The method of claim 16, wherein a target peptide representing the E. coli beta-galactosidase consists of the amino acid sequence of SEQ ID NO: 48, and mother and daughter ions thereof have mass-to-charge ratios of m/z 542.3 and m/z 636.3, respectively.

18. The method of claim 9, wherein an agent for measuring the expression level of the gene encoding the S100A8, S100A9, ANXA2, KRT19, TRX1 GSN, APOC1, CA1, CHL1, FN1, LPA, MUC1 or PLAC1 protein comprise at least one selected from the group consisting of a primer, a probe and an antisense nucleotide, which bind specifically to the gene encoding the S100A8, S100A9, ANXA2, KRT19, TRX1, GSN, APOC1, CA1, CHL1, FN1, LPA, MUC1 or PLAC1 protein.

19. The method of claim 9, wherein the measuring of the expression level of the gene encoding the S100A8, S100A9, ANXA2, KRT19, TRX1 GSN, APOC1, CA1, CHL1, FN1, LPA, MUC1 or PLAC1 protein is performed by reverse transcription-polymerase chain reaction (RT-PCR), competitive RT-PCR, real-time RT-PCR, RNase protection assay (RPA), Northern blotting, or DNA chip assay.

20. The method of claim 9, wherein, when the measured expression level of either at least one protein selected from the group consisting of S100A8, S100A9, ANXA2, KRT19, TRX1, GSN, APOC1, CA1, CHL1, FN1, LPA, MUC1 and PLAC1, or the gene encoding the protein, in the biological sample isolated from the subject of interest, increases or decreases compared to that in a normal control group, it is predicted that the subject has a high likelihood of developing the cancer.

21-24. (canceled)

25. The method of claim 9, wherein the cancer is breast cancer, ovarian cancer, colorectal cancer, stomach cancer, liver cancer, pancreatic cancer, cervical cancer, thyroid cancer, parathyroid cancer, lung cancer, non-small cell lung cancer, prostate cancer, gallbladder cancer, biliary tract cancer, non-Hodgkin's lymphoma, Hodgkin's lymphoma, blood cancer, bladder cancer, kidney cancer, melanoma, colon cancer, bone cancer, skin cancer, head cancer, uterine cancer, rectal cancer, brain tumor, perianal cancer, fallopian tube carcinoma, endometrial carcinoma, vaginal cancer, vulvar carcinoma, esophageal cancer, small intestine cancer, endocrine adenocarcinoma, adrenal cancer, soft tissue sarcoma, urethral cancer, penile cancer, ureteral cancer, renal cell carcinoma, renal pelvic carcinoma, central nervous system (CNS) tumor, primary CNS lymphoma, spinal cord tumor, brainstem glioma or pituitary adenoma.

26. A method for screening a drug for preventing or treating cancer, the method comprising steps of:
(a) treating either a sample isolated from a cancer subject or a cancer disease animal model with a candidate drug; and
(b) measuring an expression level of either at least one protein selected from the group consisting of S100A8, S100A9, ANXA2, KRT19, TRX1, GSN, APOC1, CA1, CHL1, FN1, LPA, MUC1 and PLAC1, or a gene encoding the protein, in the sample or cancer disease animal model with the candidate drug.

27. The method of claim 26, wherein the sample is cells or tissue isolated from the cancer subject.

28. The method of claim 26, further comprising step (c) of determining that the candidate drug is the drug for preventing or treating cancer, when the expression level of the S100A8, S100A9, ANXA2, KRT19, TRX1, GSN, APOC1, CA1, CHL1, FN1, LPA, MUC1 or PLAC1 protein, or the gene encoding the protein, measured in step (b), increases or decreases compared to that before treatment with the candidate drug.

29. (canceled)
